(19) United States
(12) Patent Application Publication
(10) Pub. No.: US 2012/0270848 A1
(43) Pub. Date: Oct. 25, 2012

(54) Novel Compositions and Therapeutic Methods Using Same

(75) Inventors: James C. Mannion, Plainsboro, NJ (US); Scott L. Dax, Landenberg, PA (US)

(73) Assignee: Galleon Pharmaceuticals, Inc.

(21) Appl. No.: 13/452,104

(22) Filed: Apr. 20, 2012

Related U.S. Application Data
(63) Continuation-in-part of application No. 12/910,490, filed on Oct. 22, 2010.

(51) Int. Cl. A61K 31/5377; A61P 25/20; A61P 11/00; A61K 31/57; A61P 25/04

(52) U.S. Cl. 514/171; 514/235.5

(57) ABSTRACT

The present invention includes compositions and methods for treating a subject in need of opioid therapy, wherein the opioid therapy produces or has the possibility of producing respiratory depression or a breathing control disorder in the subject.

### CROSS-REFERENCE TO RELATED APPLICATIONS

The present application is a continuation-in-part of, and claims priority to, U.S. application Ser. No. 12/910,490, filed Oct. 22, 2010, which application is incorporated by reference herein in its entirety.

### BACKGROUND OF THE INVENTION

Normal control of breathing is a complex process that involves the body's interpretation and response to chemical stimuli such as carbon dioxide, pH and oxygen levels in blood, tissues and the brain. Breathing control is also affected by wakefulness (i.e., whether the patient is awake or sleeping). Within the brain medulla, there is a respiratory control center that interprets the various signals that affect respiration and issues commands to the muscles that perform the work of breathing. Key muscle groups are located in the abdomen, diaphragm, pharynx and thorax. Sensors located centrally and peripherally then provide input to the brain's central respiration control areas that enables response to changing oxygen requirements.

Normal respiratory rhythm is maintained primarily by the body's rapid response to changes in carbon dioxide levels (CO₂). Increased CO₂ levels signal the body to increase breathing rate and depth, resulting in higher oxygen levels and subsequent lower CO₂ levels. Conversely, low CO₂ levels can result in periods of apnea (no breathing) since the stimulation to breathe is absent. This is what happens when a person hyperventilates.

In addition to the role of the brain, breathing control is the result of feedback from both peripheral and central chemoreceptors, but the exact contribution of each is unknown.

There are many diseases in which loss of normal breathing rhythm is a primary or secondary feature of the disease. Examples of diseases with a primary loss of breathing rhythm control are apneas (central, mixed or obstructive; where the breathing repeatedly stops for 10 to 60 seconds) and congenital central hypoventilation syndrome. Secondary loss of breathing rhythm may be due to chronic cardio-pulmonary diseases (e.g., heart failure, chronic bronchitis, emphysema, and impending respiratory failure), excessive weight (e.g., obesity-hypoventilation syndrome), certain drugs (e.g., anesthetics, sedatives, anxiolytics, hypnotics, alcohol, and narcotic analgesics) and/or factors that affect the neurological system (e.g., stroke, tumor, trauma, radiation damage, and ALS). In chronic obstructive pulmonary diseases where the body is exposed to chronically low levels of oxygen, the body adapts to the lower pH by a kidney mediated retention of bicarbonate, which has the effect of partially neutralizing the CO₂/pH respiratory stimulation. Thus, the patient must rely on the less sensitive oxygen-based system.

In particular, loss of normal breathing rhythm during sleep is a common condition. Sleep apnea is characterized by frequent periods of no or partial breathing. Key factors that contribute to these apneas include decrease in CO₂ receptor sensitivity, decrease in hypoxic ventilatory response sensitivity (e.g., decreased response to low oxygen levels) and loss of “wakefulness.” Normal breathing rhythm is disturbed by apnea events, resulting in hypoxia (and the associated oxidative stress) and eventually severe cardiovascular consequences (high blood pressure, stroke, heart attack). Snoring has some features in combination with sleep apnea. The upper airway muscles lose their tone resulting in the sounds associated with snoring but also inefficient airflow, which may result in hypoxia.

The ability of a mammal to breathe, and to modify breathing according to the amount of oxygen available and demands of the body, is essential for survival. There are a variety of conditions that are characterized by or due to either a primary or secondary cause. Estimates for U.S. individuals afflicted with conditions wherein there is compromised respiratory control include sleep apneas (15-20 millions); obesity-hypoventilation syndrome (5-10 millions); chronic heart disease (5 millions); chronic obstructive pulmonary disease (COPD)/chronic bronchitis (10 millions); drug-induced hypoventilation (2-5 millions); and mechanical ventilation weaning (0.5 million).

Racemic 1-ethyl-4-(2-morphilinoethyl)-3,3-diphenyl-2-pyrrolidinone (commonly known as doxapram) is a known respiratory stimulant, marketed under the name of Dopram™.

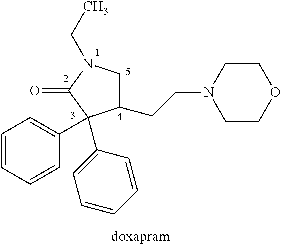

Doxapram was first synthesized in 1962 and shown to have a strong, dose-dependent effect on stimulating respiration (breathing) in animals (Ward & Franko, 1962, Fed. Proc. 21:325). Administered intravenously, doxapram causes an increase in tidal volume and respiratory rate. Doxapram is used in intensive care settings to stimulate respiration in patients with respiratory failure and to suppress shivering after surgery. Doxapram is also useful for treating respiratory depression in patients who have taken excessive doses of opioid drugs such as buprenorphine and fail to respond adequately to treatment with naloxone. However, use of doxapram in the medical setting is hampered by several reported side effects. High blood pressure, panic attacks, tachycardia (rapid heart rate), tremor, convulsions, sweating, vomiting and the sensation of “air hunger” may occur upon doxapram administration. Therefore, doxapram may not be used in patients with coronary heart disease, epilepsy and high blood pressure.

The C-4 carbon in the structure of doxapram is a chiral center, and thus there are two distinct enantiomers associated with this molecule: the (+)-enantiomer and the (−)-enantiomer. The concept of enantiomers is well known to those skilled in the art. The two enantiomers have the same molecular formula and identical chemical connectivity but opposite spatial “handedness.” The two enantiomers are a mirror image of each other but are not superimposable.

Chiral molecules have the unique property of causing a rotation in the original plane of vibration of plane-polarized light. Individual enantiomers are able to rotate plane-polarized light in a clockwise (dextrorotary; the (+)-enantiomer) or counter clockwise (levorotatory; the (−)-enantiomer) manner. For a specific combination of solvent, concentration and temperature, the pure enantiomers rotate plane-polarized light by the same number of degrees but in opposite directions.

A racemic mixture or a “racemate” is a term used to indicate the mixture of essentially equal quantities of enantiomeric pairs. Racemic mixtures are devoid of appreciable optical activity due to the mutually opposing optical activities of the individual enantiomers. Apart from their interaction with polarized light, enantiomers may differ in their physical, chemical and pharmacology activities, but such differences between enantiomers are largely unpredictable. Recent attempts have been made to develop pure enantiomers as new drugs, based on previously marketed racemic drugs (Nunez et al., 2009, Curr. Med. Chem. 16(16):2064-74). Development of an individual enantiomer as a novel drug, based on the already used racemate, requires the de novo pharmacokinetic, pharmacological and toxicological characterization of the enantiomer, since its properties may differ substantially and unpredictably from those of the racemate.

Doxapram is marketed and medically used as a racemate. Doxapram has been previously separated into its pure enantiomers using methods such as chiral high-performance chromatography (Chankvetadze et al., 1996, J. Pharm. Biomed. Anal. 14:1295-1303; Thunberg et al., 2002, J. Pharm. Biomed. Anal. 27:431-39), and chiral capillary electrophoresis (Christians & Holzgrabe, 2001, J. Chromat. A 911:249-57). Using in silico methods, the enantiomers of doxapram were predicted to have identical oral bioavailability (Moda et al., 2007, Bioorg. Med. Chem. 15:7738-45).

There is a need in the art for a method of treating breathing control disorders or diseases. Such method should include the administration of a composition comprising a compound that restores all or part of the body's normal breathing control system in response to changes in CO₂ and/or oxygen, and yet has minimal side effects. There is a further need for compositions and methods useful for treating a subject in need of opioid therapy, wherein opioid therapy produces or has the possibility of producing respiratory depression or a breathing control disorder. The present invention fulfills these needs.

### BRIEF SUMMARY OF THE INVENTION

The invention includes a method of treating a subject in need of opioid therapy, wherein the opioid therapy produces or has the possibility of producing respiratory depression or a breathing control disorder. The method comprises administering to the subject an effective amount of a pharmaceutical composition comprising a compound selected from the group consisting of (+)-doxapram, a deuterated derivative thereof, any salt thereof, and any combinations thereof. The method further comprises administering to the subject an effective amount of an opioid, wherein the composition is essentially free of (−)-doxapram, a deuterated derivative thereof, a salt thereof or any combinations thereof.

In one embodiment, the compound is at least about 95% enantiomerically pure. In another embodiment, the compoundis at least about 97% enantiomerically pure. In yet another embodiment, the compound is at least about 99% enantiomerically pure. In yet another embodiment, the opioid comprises morphine, codeine, heroin, hydromorphone, hydrocodone, oxymorphone, oxycodone, meperidine, methadone, nalbuphine, butorphanol, buprenorphine, propoxyphene, pentazocine, dihydrocodeine, tapentadol, fentanyl, remifentanil, alfentanil, sufentanil, carfentanil, or any combinations thereof. In yet another embodiment, the subject is further administered at least one additional compound selected from the group consisting of acetazolamide, almitrine, theophylline, caffeine, methyl progesterone, a serotinergic modulator, an ampakine, and any combinations thereof. In yet another embodiment, the composition is administered in conjunction with the use of a mechanical ventilation device or positive airway pressure device on the subject. In yet another embodiment, the composition is administered to the subject by an inhalational, topical, oral, buccal, rectal, vaginal, intramuscular, subcutaneous, transdermal, intrathecal or intravenous route. In yet another embodiment, the administering of the compound takes place before or after the administering of the opioid to the subject. In yet another embodiment, the administering of the compound takes place within 6 hours of the administering of the opioid to the subject. In yet another embodiment, the compoundand the opioid are co-administered to the subject. In yet another embodiment, the compound and the opioid are co-formulated. In yet another embodiment, the composition further comprises a pharmaceutically acceptable carrier. In yet another embodiment, the subject is a mammal. In yet another embodiment, the mammal is human.

The invention also includes a pharmaceutical composition comprising a pharmaceutically acceptable carrier, an opioid and a compound selected from the group consisting of (+)-doxapram, a deuterated derivative thereof, any salt thereof, and any combinations thereof, wherein the composition is essentially free of (−)-doxapram, a deuterated derivative thereof, a salt thereof, or any combinations thereof. In one embodiment, the compound is at least about 95% enantiomerically pure. In another embodiment, the compound is at least about 97% enantiomerically pure. In yet another embodiment, the compound is at least about 99% enantiomerically pure. In yet another embodiment, the opioid comprises morphine, codeine, heroin, hydromorphone, hydrocodone, oxymorphone, oxycodone, meperidine, methadone, nalbuphine, butorphanol, buprenorphine, propoxyphene, pentazocine, dihydrocodeine, tapentadol, fentanyl, remifentanil, alfentanil, sufentanil, carfentanil, or any combinations thereof.

The invention also includes a method of preventing or treating a breathing control disorder or disease in a subject in need thereof. The method comprises administering to the subject an effective amount of a pharmaceutical composition comprising a pharmaceutically acceptable carrier and a deuterated derivative of (+)-doxapram orany salt thereof, wherein the composition is essentially free of a deuterated derivative of (−)-doxapram or any salt thereof.

In one embodiment, the deuterated derivative of (+)-doxapram or salt thereof is at least about 95% enantiomerically pure. In another embodiment, the deuterated derivative of (+)-doxapram or salt thereof is at least about 97% enantiomerically pure. In yet another embodiment, the deuterated derivative of (+)-doxapram or salt thereof is at least about 99% enantiomerically pure. In yet another embodiment, the breathing control disorder or disease is selected from the group consisting of respiratory depression, sleep apnea, apnea of prematurity, obesity-hypoventilation syndrome, primary alveolar hypoventilation syndrome, dyspnea, hypoxia, and hypercapnia. In yet another embodiment, the subject is further administered at least one additional compound useful for treating the breathing control disorder or disease. In yet another embodiment, the at least one additional compound is selected from the group consisting of acetazolamide, almitrine, theophylline, caffeine, methyl progesterone and related compounds, a serotinergic modulator and an ampakine. In yet another embodiment, the composition is administered in conjunction with the use of a mechanical ventilation device or positive airway pressure device on the subject. In yet another embodiment, the subject is a human. In yet another embodiment, wherein the composition is administered to the subject by an inhalational, topical, oral, buccal, rectal, vaginal, intramuscular, subcutaneous, transdermal, intrathecal or intravenous route.

The invention also includes a method of preventing destabilization or stabilizing breathing rhythm in a subject in need thereof. The method comprises administering to the subject an effective amount of a pharmaceutical composition comprising a pharmaceutically acceptable carrier and a deuterated derivative of (+)-doxapram or a salt thereof, wherein the composition is essentially free of a deuterated derivative of (−)-doxapram or a salt thereof.

In one embodiment, the deuterated derivative of (+)-doxapram or a salt thereof is at least about 95% enantiomerically pure. In another embodiment, the deuterated derivative of (+)-doxapram or salt thereof is at least about 97% enantiomerically pure. In yet another embodiment, the deuterated derivative of (+)-doxapram or salt thereof is at least about 99% enantiomerically pure. In yet another embodiment, the subject is further administered at least one additional compound useful for preventing destabilization of or stabilizing the breathing rhythm. In yet another embodiment, the at least one additional compound is selected from the group consisting of acetazolamide, almitrine, theophylline, caffeine, a serotinergic modulator and an ampakine. In yet another embodiment, the composition is administered in conjunction with the use of a mechanical ventilation device or positive airway pressure device. In yet another embodiment, the subject is a mammal. In yet another embodiment, the mammal is human. In yet another embodiment, the composition is administered to the subject by an inhalational, topical, oral, buccal, rectal, vaginal, intramuscular, subcutaneous, transdermal, intrathecal or intravenous route.

The invention also includes a pharmaceutical composition comprising a pharmaceutically acceptable carrier and a deuterated derivative of (+)-doxapram or any salt thereof, wherein the composition is essentially free of a deuterated derivative of (−)-doxapram or a salt thereof. In one embodiment, the deuterated derivative of (+)-doxapram or salt thereof is at least about 95% enantiomerically pure. In another embodiment, the deuterated derivative of (+)-doxapram or salt thereof is at least about 97% enantiomerically pure. In yet another embodiment, the deuterated derivative of (+)-doxapram or salt thereof is at least about 99% enantiomerically pure.

### BRIEF DESCRIPTION OF THE DRAWINGS

For the purpose of illustrating the invention, there are depicted in the drawings certain embodiments of the invention. However, the invention is not limited to the precise arrangements and instrumentalities of the embodiments depicted in the drawings.

FIG. 1 is a graph illustrating the minute ventilation (in ml/min units), as indicated by the maximum peak response, for different intravenous doses of (+)-doxapram, (−)-doxapram and racemic doxapram.

FIG. 2 is a graph illustrating the effects of (+)-doxapram, (−)-doxapram and a vehicle control on opioid-induced respiratory depression, measured as minute ventilation (ml/min), in the rat. The opioid used was morphine.

FIG. 3 is a graph illustrating the pCO₂ (in mm Hg) in the rat upon administration of morphine (10 mg/kg) followed by an infusion of (curve A) vehicle, (curve B) (−)-doxapram, (curve C) (+)-doxapram, or (curve D) racemic doxapram. The infusion duration is indicated by the bar.

FIG. 4 is a graph illustrating the O₂ saturation (in %) in the rat upon administration of morphine (10 mg/kg) followed by an infusion of (curve A) vehicle, (curve B) (−)-doxapram, (curve C) (+)-doxapram, or (curve D) racemic doxapram. The infusion duration is indicated by the bar.

FIG. 5 is a graph illustrating the effects of (+)-doxapram, (−)-doxapram and a vehicle control on the hypoxic ventilatory response, measured as minute ventilation (ml/min), to 12% O₂ in the rat.

FIG. 6, comprising FIGS. 6A-6B, is a series of traces illustrating the effects of 30 mg/kg IV (+)-doxapram on (from top to bottom in FIG. 6A) respiratory flow (in ml/min), blood pressure (in mm Hg), inspiratory volume (in ml/min), and (from top to bottom in FIG. 6B) expiratory volume (in ml/min), respiratory rate (in breaths/min), and minute ventilation (in ml/min) in the rat. The y-axis indicates the parameter in question, and the x-axis indicates time (in min). The dotted line indicates IV bolus administration (30 mg/kg) of (+)-doxapram.

FIG. 7, comprising FIGS. 7A-7B, is a series of traces illustrating the effects of 30 mg/kg IV (−)-doxapram (from top to bottom in FIG. 7A) respiratory flow (in ml/min), blood pressure (in mm Hg), inspiratory volume (in ml/min), and (from top to bottom in FIG. 7B) expiratory volume (in ml/min), respiratory rate (in breaths/min), and minute ventilation (in ml/min) in the rat. The y-axis indicates the parameter in question, and the x-axis is time (min). The dotted line indicates IV bolus administration (30 mg/kg) of (−)-doxapram.

FIG. 8 is a graph illustrating the effects of (−)-doxapram on blood pressure (in mm Hg) in the rat (as a detail enlargement of the corresponding curve illustrated in FIG. 7). The y-axis is blood pressure, and the x-axis is time. The dotted line indicates start of administration of (−)-doxapram (30 mg/kg IV bolus).

FIG. 9 is a set of graphs illustrating the IV pharmacokinetics of a 20-minute infusion (from 15 minutes to 35 minutes) of 3 mg/kg/min IV (+)-doxapram and (−)-doxapram. The upper panel pharmacokinetic data was plotted on a linear y-axis, the lower panel represents the same data plotted on a log y-axis. The plasma exposures of the two enantiomers have directly comparable time course, maximum concentration and exposure (AUC), thus demonstrating there is no appreciable difference between the pharmacokinetics properties of the two enantiomers.

### DETAILED DESCRIPTION OF THE INVENTION

In one aspect, the present invention relates to the unexpected discovery that the (+)-enantiomer of doxapram or a deuterated derivative thereof displays most or all the desired beneficial pharmacological activity associated with the racemic doxapram (which is marketed and used for the treatment of respiratory diseases and disorders).

In another aspect, the present invention relates to the unexpected discovery that the (−)-enantiomer of doxapram or a deuterated derivative thereof is essentially devoid of activity in stimulating ventilation or reversing respiratory depression, and moreover produces a number of acute side effects that were not detected as the same doses with (+)-doxapram or a deuterated derivative thereof, such as hunching posture, increased urination and defecation, clonic movements and other seizure-like behaviors, pronounced drops in mean arterial blood pressure, and production of cardiac arrhythmias and death.

The present invention includes a pharmaceutical composition comprising the (+)-enantiomer of 1-ethyl-4-(2-morphilinoethyl)-3,3-diphenyl-2-pyrrolidinone, also known as (+)-doxapram, a deuterated derivative thereof, or a salt thereof and a pharmaceutically acceptable carrier, wherein the composition is essentially free of (−)-doxapram, a deuterated derivative thereof, or a salt thereof.

The present invention also includes a method of treating a breathing control disease or disorder in a subject in need thereof. The breathing control disease or disorder includes, but is not limited to, respiratory depression (induced by anesthetics, sedatives, anxiolytic agents, hypnotic agents, alcohol, and analgesics), sleep apnea, apnea of prematurity, obesity-hypoventilation syndrome, primary alveolar hypoventilation syndrome, dyspnea, hypoxia and hypercapnia. The method comprises administering to the subject a therapeutically effective amount of a pharmaceutical composition comprising (+)-doxapram, a deuterated derivative thereof, or a salt thereof, and a pharmaceutically acceptable carrier, wherein the composition is essentially free of (−)-doxapram, a deuterated derivative thereof, or a salt thereof.

The present invention also includes a method of treating a subject in need of opioid therapy, wherein opioid therapy produces or has the possibility of producing respiratory depression or a breathing control disorder, wherein the subject is administered an opioid and a composition comprising (+)-doxapram, a deuterated derivative thereof, a salt thereof, or any mixtures thereof, wherein the composition is essentially free of (−)-doxapram, a deuterated derivative thereof, a salt thereof, or any mixtures thereof. In one embodiment, the opioid and (+)-doxapram, or deuterated derivative thereof, are administered separately to the subject. In another embodiment, the opioid and (+)-doxapram, or deuterated derivative thereof, are co-administered to the subject. In yet one embodiment, the opioid and (+)-doxapram, or deuterated derivative thereof, are co-formulated and co-administered to the subject.

### Definitions

As used herein, each of the following terms has the meaning associated with it in this section.

Unless defined otherwise, all technical and scientific terms used herein generally have the same meaning as commonly understood by one of ordinary skill in the art to which this invention belongs. Generally, the nomenclature used herein and the laboratory procedures in cell culture, animal pharmacology, and organic chemistry are those well-known and commonly employed in the art.

As used herein, the articles “a” and “an” refer to one or to more than one (i.e. to at least one) of the grammatical object of the article. By way of example, “an element” means one element or more than one element.

A “subject”, as used therein, can be a human or non-human mammal. Non-human mammals include, for example, livestock and pets, such as ovine, bovine, porcine, canine, feline and murine mammals. Preferably, the subject is human.

As used herein, the term “doxapram” refers to 1-ethyl-4-(2-morphilinoethyl)-3,3-diphenyl-2-pyrrolidinone, or a salt thereof. Unless otherwise noted, “doxapram” refers to racemic doxapram, which comprises an essentially equimolar mixture of the two enantiomers of doxapram (the (+)-enantiomer and the (−)-enantiomer).

As used herein, the “(+)-doxapram” and “(−)-doxapram” enantiomers are defined in terms of the order in which they are eluted from chiral HPLC column, defined as: (a) a CHIRALPAK® AY 20μ column, with 3 cm internal diameter×25 cm length, using ethanol with 0.2% DMEA (dimethylethylamine) and CO₂ as mobile phase, in a ratio of 15:85, with a flow rate of 85 g/min, a column temperature of 35° C., and UV detection at 220 nm; or (b) a CHIRALPAK® AY-H 5μ column, with 3 cm internal diameter×25 cm length, using ethanol with 0.2% DMEA and CO₂ as mobile phase, in a ratio of 15:85, with a flow rate of 85 g/min, a column temperature of 35° C., and UV detection at 220 nm. Under either condition, the (−)-doxapram enantiomer has a shorter elution/retention time from the column than the (+)-doxapram enantiomer. The nomenclature “(+)-doxapram” should not be construed to imply that this enantiomer rotates the vibrational plane of plane-polarized light in a clockwise manner under all possible combinations of solvent, temperature and concentration. Similarly, the nomenclature “(−)-doxapram” should not be construed to imply that this enantiomer rotates the vibrational plane of plane-polarized light in a counter-clockwise manner under all possible combinations of solvent, temperature and concentration.

As used herein, the term “enantiomeric purity” of a given enantiomer over the opposite enantiomer indicates the excess % of the given enantiomer over the opposite enantiomer, by weight. For example, in a mixture comprising about 80% of a given enantiomer and about 20% of the opposite enantiomer, the enantiomeric purity of the given enantiomer is about 60%.

As used herein, the term “essentially free of” as applied to a given enantiomer in a mixture with the opposite enantiomer indicates that the enantiomeric purity of the given enantiomer is higher than about 80%, more preferably higher than about 90%, even more preferably higher than about 95%, even more preferably higher than about 97%, even more preferably higher than about 99%, even more preferably higher than about 99.5%, even more preferably higher than about 99.9%, even more preferably higher than about 99.95%, even more preferably higher than about 99.99%. Such purity determination may be made by any method known to those skilled in the art, such as chiral HPLC analysis or chiral electrophoresis analysis.

In a non-limiting embodiment, the following terminology used to report blood gas measurements is well known to those skilled in the art and may be defined as such: minute ventilation (MV) is a measure of breathing volume per unit time and is given herein as ml/min; pCO₂ is partial pressure of carbon dioxide (gas) in (arterial) blood measured in mmHg (millimeters of Hg units); pO₂ is partial pressure of oxygen (gas) in (arterial) blood measured in mmHg (millimeters of Hg units); saO₂ is the percentage of oxygen saturation (dissolved oxygen gas) which correlates to the percentage of hemoglobin binding sites in the bloodstream occupied by oxygen.

As used herein, the term ED₅₀ refers to the effective dose that produces a given effect in 50% of the subjects.

As used herein, a “disease” is a state of health of an animal wherein the animal cannot maintain homeostasis, and wherein if the disease is not ameliorated then the animal's health continues to deteriorate.

As used herein, a “disorder” in an animal is a state of health in which the animal is able to maintain homeostasis, but in which the animal's state of health is less favorable than it would be in the absence of the disorder. Left untreated, a disorder does not necessarily cause a further decrease in the animal's state of health.

As used herein, an “effective amount” or “therapeutically effective amount” of a compound is that amount of compound which is sufficient to provide a beneficial effect to the subject to which the compound is administered. The term to “treat,” as used herein, means reducing the frequency with which symptoms are experienced by a patient or subject or administering an agent or compound to reduce the severity with which symptoms are experienced.

As used herein, “treating a disease or disorder” means reducing the frequency with which a symptom of the disease or disorder is experienced by a patient. Disease and disorder are used interchangeably herein.

As used herein, the term “adverse events” (AEs) or “adverse effects” refer to a change in normal behavior or homeostasis and refers to observed or measured effects in animals such as hunching posture, increased urination and defecation, clonic movements and other seizure-like behaviors, pronounced drops in mean arterial blood pressure, production of cardiac arrhythmias and death. Adverse effects or adverse events may also refer to respiratory depression caused by opioids.

As used herein, the term “about” will be understood by persons of ordinary skill in the art and will vary to some extent on the context in which it is used. As used herein when referring to a measurable value such as an amount, a temporal duration, and the like, the term “about” is meant to encompass variations of ±20% or ±10%, more preferably ±5%, even more preferably ±1%, and still more preferably ±0.1% from the specified value, as such variations are appropriate to perform the disclosed methods.

### Compositions of the Invention

The invention includes a pharmaceutical composition comprising a pharmaceutically acceptable carrier and (+)-doxapram, a deuterated derivative thereof, or a salt thereof, wherein the composition is essentially free of (−)-doxapram, a deuterated derivative thereof, or a salt thereof.

The invention also includes a pharmaceutical composition comprising a pharmaceutically acceptable carrier, an opioid and (+)-doxapram, a deuterated derivative thereof, or a salt thereof, wherein the composition is essentially free of (−)-doxapram, a deuterated derivative thereof, or a salt thereof.

In one embodiment, the (+)-doxapram, deuterated derivative thereof, or salt thereof is at least about 95% enantiomerically pure. In another embodiment, the (+)-doxapram, deuterated derivative thereof, or salt thereof is at least about 97% enantiomerically pure. In yet another embodiment, the (+)-doxapram, deuterated derivative thereof, or salt thereof is at least about 99% enantiomerically pure. In yet another embodiment, the opioid comprises morphine, codeine, heroin, hydromorphone, hydrocodone, oxymorphone, oxycodone, meperidine, methadone, nalbuphine, butorphanol, buprenorphine, propoxyphene, pentazocine, dihydrocodeine, tapentadol, fentanyl, remifentanil, alfentanil, sufentanil, carfentanil, or any combinations thereof.

Racemic doxapram or a salt thereof may be prepared using any of the methods disclosed in the chemical literature. As a non-limiting example, the synthetic scheme illustrated below may be used to prepare racemic doxapram.

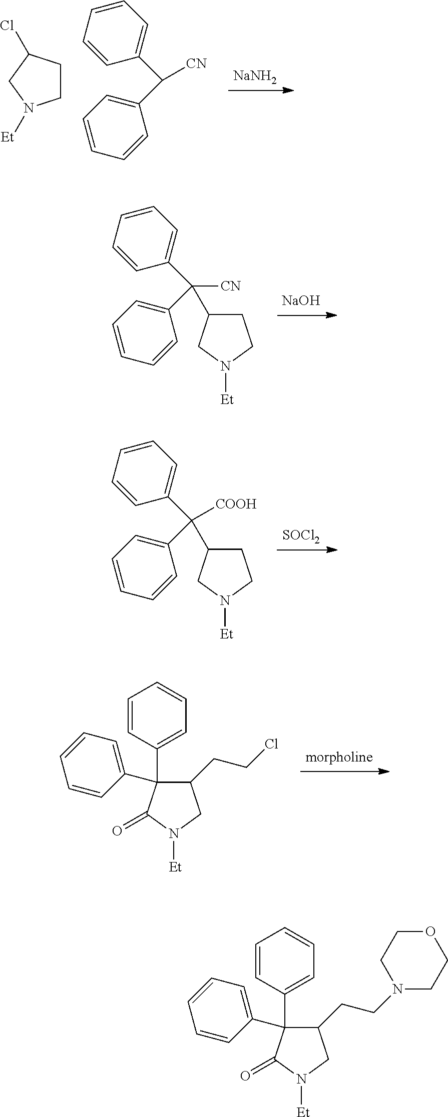

(+)-Doxapram, deuterated derivative thereof or a salt thereof that is essentially free of (−)-doxapram, a deuterated derivative thereof or a salt thereof may be prepared by chiral resolution of racemic doxapram, using a method such as chiral chromatography (in a non-limiting example, chiral HPLC). In a non-limiting example, (+)-doxapram or a salt thereof, which is essentially free of (−)-doxapram or a salt thereof, may be isolated from racemic doxapram in >99% enantiomeric excess using supercritical fluid chromatography (SFC) and a suitable chiral column, such as a CHIRALPAK® AY, 20μ (micron), 30×250 mm column with EtOH with 0.2% DMEA (dimethylethylamine) and CO₂ (15:85) as mobile phase. Alternatively, the same separation may be performed on a CHIRALPAK® AY-H, 5μ column, 4.6×250 mm column with EtOH with 0.2% DMEA:CO₂ (15:85) as mobile phase. Doxapram enantiomers may also be analyzed using a CHIRALCEL® OJ-H, 5μ with 90% hexane—8% isopropanol—2% methanol—0.1% DMEA. The columns are operated according to the manufacturer's instructions.

### Methods of the Invention

A composition comprising (+)-doxapram, a deuterated derivative thereof, or a salt thereof, wherein the composition is essentially free of (−)-doxapram, a deuterated derivative thereof, or a salt thereof, is useful within the methods of the invention. A composition comprising an opioid and (+)-doxapram, a deuterated derivative thereof, or salt thereof, wherein the composition is essentially free of (−)-doxapram, deuterated derivative thereof, or salt thereof, is also useful within the methods of the invention.

In one aspect, the present invention relates to the unexpected discovery that the (+)-enantiomer of doxapram, deuterated derivative thereof, or salt thereof displays most or all the desired beneficial pharmacological activity associated with the ventilatory stimulant effects, and positive effects on arterial blood gases, of racemic doxapram (which is marketed and used for the treatment of respiratory diseases and disorders).

In another aspect, the present invention relates to the unexpected discovery that the (−)-enantiomer of doxapram, deuterated derivative thereof, or salt thereof is essentially devoid of activity as a ventilatory or respiratory stimulant, but unexpectedly produces adverse side effects, such as hunching posture, increased urination and defecation, clonic movements and other seizure-like behaviors, pronounced drops in mean arterial blood pressure, production of cardiac arrhythmias and death.

The invention includes a method of treating a subject in need of opioid therapy, wherein opioid therapy produces or has the possibility of producing respiratory depression or a breathing control disorder. The method comprises administering to the subject an effective amount of a pharmaceutical composition comprising (+)-doxapram, a deuterated derivative thereof, or a salt thereof. The method further comprises administering to the subject an effective amount of an opioid, wherein the composition is essentially free of (−)-doxapram, a deuterated derivative thereof, or a salt thereof.

In one embodiment, the (+)-doxapram, deuterated derivative thereof, or salt thereof is at least about 95% enantiomerically pure. In another embodiment, the (+)-doxapram, deuterated derivative thereof, or salt thereof is at least about 97% enantiomerically pure. In yet another embodiment, the (+)-doxapram, deuterated derivative thereof, or salt thereof is at least about 99% enantiomerically pure. In yet another embodiment, the opioid comprises morphine, codeine, heroin, hydromorphone, hydrocodone, oxymorphone, oxycodone, meperidine, methadone, nalbuphine, butorphanol, buprenorphine, propoxyphene, pentazocine, dihydrocodeine, tapentadol, fentanyl, remifentanil, alfentanil, sufentanil, carfentanil, or any combinations thereof. In yet another embodiment, the subject is further administered at least one additional compound selected from the group consisting of acetazolamide, almitrine, theophylline, caffeine, methyl progesterone, a serotinergic modulator, an ampakine, and any combinations thereof. In yet another embodiment, the composition is administered in conjunction with the use of a mechanical ventilation device or positive airway pressure device on the subject. In yet another embodiment, the composition is administered to the subject by an inhalational, topical, oral, buccal, rectal, vaginal, intramuscular, subcutaneous, transdermal, intrathecal or intravenous route. In another embodiment, the composition further comprises a pharmaceutically acceptable carrier. In yet another embodiment, the subject is a mammal. In yet another embodiment, the mammal is human.

The invention contemplates administering the opioid and (+)-doxapram, or deuterated derivative thereof, to the subject separately (i.e., the administering of (+)-doxapram or a salt thereof takes place before or after the administering of the opioid to the subject). In one embodiment, the administering of (+)-doxapram, deuterated derivative thereof, or salt thereof takes place within 6 hours of the administering of the opioid to the subject. In another embodiment, the administering of (+)-doxapram, deuterated derivative thereof, or salt thereof takes place within 5 hours of the administering of the opioid to the subject. In yet another embodiment, the administering of (+)-doxapram, deuterated derivative thereof, or salt thereof takes place within 4 hours of the administering of the opioid to the subject. In another embodiment, the administering of (+)-doxapram, deuterated derivative thereof, or salt thereof takes place within 3 hours of the administering of the opioid to the subject. In another embodiment, the administering of (+)-doxapram, deuterated derivative thereof, or salt thereof takes place within 2 hours of the administering of the opioid to the subject. In another embodiment, the administering of (+)-doxapram, deuterated derivative thereof, or salt thereof takes place within 1 hour of the administering of the opioid to the subject. In another embodiment, the administering of (+)-doxapram, deuterated derivative thereof, or salt thereof takes place within 45 minutes of the administering of the opioid to the subject. In another embodiment, the administering of (+)-doxapram, deuterated derivative thereof, or salt thereof takes place within 30 minutes of the administering of the opioid to the subject. In another embodiment, the administering of (+)-doxapram, deuterated derivative thereof, or salt thereof takes place within 15 minutes of the administering of the opioid to the subject. In another embodiment, the administering of (+)-doxapram, deuterated derivative thereof, or salt thereof takes place within 5 minutes of the administering of the opioid to the subject.

The invention also contemplates co-administering (+)-doxapram, deuterated derivative thereof, or salt thereof and the opioid to the subject. In one embodiment, the (+)-doxapram, deuterated derivative thereof, or salt thereof and the opioid are co-formulated. (+)-Doxapram, deuterated derivative thereof, or salt thereof and the opioid may be co-formulated using any pharmaceutically acceptable carrier known to those skilled in the art.

The experiments disclosed in the present invention suggest that a composition comprising (+)-doxapram, deuterated derivative thereof, or salt thereof, wherein the composition is essentially free of (−)-doxapram, deuterated derivative thereof, or salt thereof, may be administered to a subject who is prone to or suffers from a breathing control disorder or disease in order to prevent, treat or mitigate the breathing control disorder. Administration of a composition comprising (+)-doxapram, deuterated derivative thereof, or salt thereof, wherein the composition is essentially free of (−)-doxapram, deuterated derivative thereof, r a salt thereof, is unexpectedly advantageous over administration of racemic doxapram or a salt thereof, because (+)-doxapram, deuterated derivative thereof, or salt thereof has most or all the desired beneficial pharmacological respiratory stimulant activity, together with positive effects on arterial blood gases, associated with racemic doxapram but with significantly reduced adverse side effects compared to administration of racemic doxapram or a salt thereof, due to the presence of the (−)-enantiomer, which has no specific ventilatory activity but produces side effects and toxicity.

In one aspect, the present invention includes a method of preventing or treating a breathing control disorder or disease in a subject in need thereof. The method comprises administering to the subject an effective amount of a pharmaceutical composition comprising (+)-doxapram, deuterated derivative thereof, or salt thereof and a pharmaceutically acceptable carrier, wherein the composition is essentially free of (−)-doxapram, deuterated derivative thereof, or a salt thereof.

In one embodiment, the enantiomeric purity of the (+)-doxapram, deuterated derivative thereof, or salt thereof is at least about 90%. In another embodiment, the enantiomeric purity of the (+)-doxapram, deuterated derivative thereof, or salt thereof is at least about 95%. In yet another embodiment, the enantiomeric purity of the (+)-doxapram, deuterated derivative thereof, or salt thereof is at least about 97%. In yet another embodiment, the enantiomeric purity of the (+)-doxapra, deuterated derivative thereof, or salt thereof is at least about 99%. In yet another embodiment, the enantiomeric purity of the (+)-doxapram, deuterated derivative thereof, or salt thereof is at least about 99.5%. In yet another embodiment, the enantiomeric purity of the (+)-doxapram, deuterated derivative thereof, or salt thereof is at least about 99.9%. In yet another embodiment, the enantiomeric purity of the (+)-doxapram, deuterated derivative thereof, or a salt thereof is at least about 99.95%. In yet another embodiment, the enantiomeric purity of the (+)-doxapram, deuterated derivative thereof, or salt thereof is at least about 99.99%.

The invention also contemplates a pharmaceutical composition comprising a deuterated derivative of (+)-doxapram or a salt thereof, wherein the composition is essentially free of (−)-doxapram, a deuterated derivative of (−)-doxapram, or a salt thereof. In one embodiment, the deuterated derivative of (+)-doxapram has better or equivalent properties as compared to (+)-doxapram, such as but not limited to pharmacokinetics, absorption, metabolism, activity or side effects. The invention contemplates any deuterated derivative of (+)-doxapram, varying from 1-30 deuteriums. For example, the deuterated derivative of (+)-doxapram may have 0-5 deuteriums in the ethyl group, 0-10 deuteriums on the phenyl groups, 0-3 deuteriums on the pyrrolidinone ring, 0-4 deuteriums in the ethylene linker, and 0-8 deuteriums on the morpholino group.

The invention also includes a pharmaceutical composition comprising a pharmaceutically acceptable carrier and a deuterated derivative of (+)-doxapram or any salt thereof, wherein the composition is essentially free of a deuterated derivative of (−)-doxapram or a salt thereof. In one embodiment, the deuterated derivative of (+)-doxapram or any salt thereof is at least about 95% enantiomerically pure. In another embodiment, the deuterated derivative of (+)-doxapram or any salt thereof is at least about 97% enantiomerically pure. In yet another embodiment, the deuterated derivative of (+)-doxapram or any salt thereof is at least about 99% enantiomerically pure.

Non-limiting examples of deuterated derivatives of doxapram useful within the compositions and methods of the invention are illustrated below:

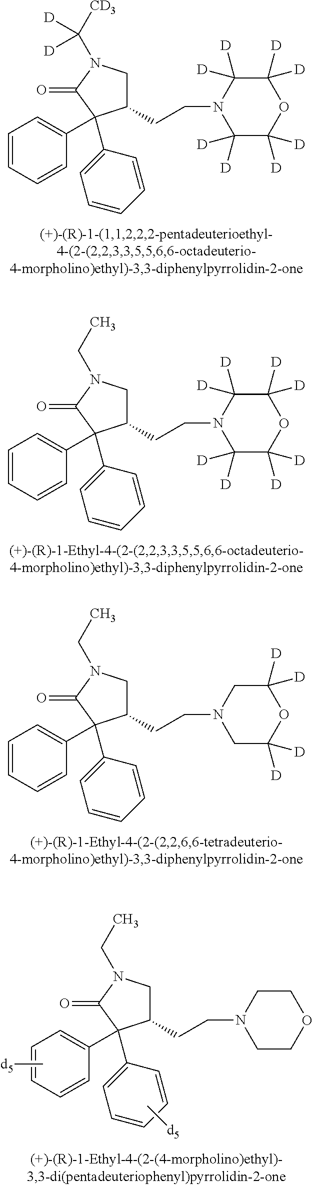

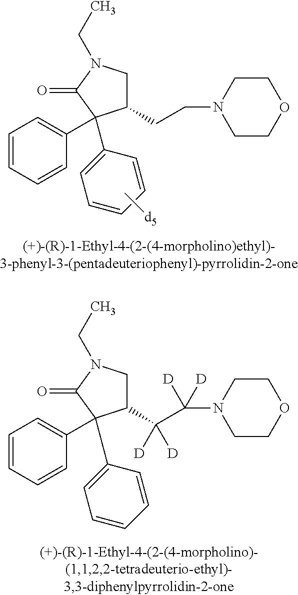

In one embodiment, the breathing control disorder or disease is selected from the group consisting of narcotic-induced respiratory depression, sleep apnea, apnea of prematurity, obesity-hypoventilation syndrome, primary alveolar hypoventilation syndrome, dyspnea, hypoxia and hypercapnia. In yet another embodiment, the subject is further administered at least one additional compound useful for treating the breathing control disorder or disease. In yet another embodiment, the at least one additional compound is selected from the group consisting of acetazolamide, almitrine, theophylline, caffeine, methylprogesterone and related compounds, a serotinergic modulator and an ampakine. In yet another embodiment, the composition is administered to the subject in conjunction with the use of a mechanical ventilation device or positive airway pressure device. In another embodiment, the subject is a human. In yet another embodiment, the composition is administered to the subject by an inhalational, topical, oral, rectal, vaginal, intramuscular, subcutaneous, transdermal, intrathecal or intravenous route.

In another aspect, the present invention includes a method of preventing destabilization of or stabilizing breathing rhythm in a subject in need thereof. The method comprises administering to the subject an effective amount of a pharmaceutical composition comprising (+)-doxapram, a deuterated derivative thereof, or a salt thereof and a pharmaceutically acceptable carrier, wherein the composition is essentially free of (−)-doxapram or a salt thereof.

In one embodiment, the enantiomeric purity of the (+)-doxapram, deuterated derivative thereof or salt thereof is at least about 90%. In another embodiment, the enantiomeric purity of the (+)-doxapram, deuterated derivative thereof or salt thereof is at least about 95%. In yet another embodiment, the enantiomeric purity of the (+)-doxapram, deuterated derivative thereof or salt thereof is at least about 97%. In yet another embodiment, the enantiomeric purity of the (+)-doxapram, deuterated derivative thereof or salt thereof is at least about 99%. In yet another embodiment, the enantiomeric purity of the (+)-doxapram, deuterated derivative thereof or salt thereof is at least about 99.5%. In yet another embodiment, the enantiomeric purity of the (+)-doxapram, deuterated derivative thereof or salt thereof is at least about 99.9%. In yet another embodiment, the enantiomeric purity of the (+)-doxapram, deuterated derivative thereof or salt thereof is at least about 99.95%. In yet another embodiment, the enantiomeric purity of the (+)-doxapram, deuterated derivative thereof or salt thereof is at least about 99.99%.

In one embodiment, the subject is further administered at least one additional compound useful for preventing destabilization of or stabilizing the breathing rhythm. In yet another embodiment, the at least one additional compound is selected from the group consisting of acetazolamide, almitrine, theophylline, caffeine, methylprogesterone and related compounds, a serotinergic modulator and an ampakine. In another embodiment, the composition is administered to the subject in conjunction with the use of a mechanical ventilation device or positive airway pressure device. In yet another embodiment, the subject is a mammal including but not limited to a human, mouse, rat, ferret, guinea, pig, monkey, dog, cat, horse, cow, pig and other farm animals. In yet another embodiment, the subject is a human. In yet another embodiment, the composition is administered to the subject by an inhalational, topical, oral, rectal, vaginal, intramuscular, subcutaneous, transdermal, intrathecal or intravenous route.

### Salts

The compounds described herein may form salts with acids, and such salts are included in the present invention. In one embodiment, the salts are pharmaceutically acceptable salts. The term “salts” embraces addition salts of free acids that are useful within the methods of the invention. The term “pharmaceutically acceptable salt” refers to salts that possess toxicity profiles within a range that affords utility in pharmaceutical applications. Pharmaceutically unacceptable salts may nonetheless possess properties such as high crystallinity, which have utility in the practice of the present invention, such as for example utility in process of synthesis, purification or formulation of compounds useful within the methods of the invention.

Suitable pharmaceutically acceptable acid addition salts may be prepared from an inorganic acid or from an organic acid. Examples of inorganic acids include hydrochloric, hydrobromic, hydriodic, nitric, carbonic, sulfuric, and phosphoric acids. Appropriate organic acids may be selected from aliphatic, cycloaliphatic, aromatic, araliphatic, heterocyclic, carboxylic and sulfonic classes of organic acids, examples of which include formic, acetic, propionic, succinic, glycolic, gluconic, lactic, malic, tartaric, citric, ascorbic, glucuronic, maleic, fumaric, pyruvic, aspartic, glutamic, benzoic, anthranilic, 4-hydroxybenzoic, phenylacetic, mandelic, embonic (pamoic), methanesulfonic, ethanesulfonic, benzenesulfonic, pantothenic, trifluoromethanesulfonic, 2-hydroxyethanesulfonic, p-toluenesulfonic, sulfanilic, cyclohexylaminosulfonic, stearic, alginic, β-hydroxybutyric, salicylic, galactaric and galacturonic acid.

### Combination Therapies.

In one embodiment, the compound (+)-doxapram, deuterated derivative thereof or salt thereof is useful in the methods of present invention in combination with at least one additional compound useful for treating breathing control disorders. These additional compounds may comprise compounds of the present invention or compounds, e.g., commercially available compounds, known to treat, prevent, or reduce the symptoms of breathing control disorders. In embodiment, the combination of the compound (+)-doxapram, deuterated derivative thereof or salt thereof and at least one additional compound useful for treating breathing control disorders has additive, complementary or synergistic effects in the treatment of disordered breathing, and in the treatment of sleep-related breathing control disorders.

In a non-limiting example, the compound (+)-doxapram, deuterated derivative thereof or a salt thereof may be used in combination with one or more of the following drugs: acetazolamide, almitrine, theophylline, caffeine, methylprogesterone and related compounds, serotinergic modulators and compounds known as ampakines. Non-limiting examples of ampakines are the pyrrolidine derivative racetam drugs such as piracetam and aniracetam; the “CX-” series of drugs which encompass a range of benzoylpiperidine and benzoylpyrrolidine structures, such as CX-516 (6-(piperidin-1-yl-carbonyl)quinoxaline), CX-546 (2,3-dihydro-1,4-benzodioxin-7-yl-(1-piperidyl)methanone), CX-614 (2H,3H,6aH-pyrrolidino(2,1-3′,2′)-1,3-oxazino-(6′,5′-5,4)benzo(e)1,4-dioxan-10-one), CX-691 (2,1,3-benzoxadiazol-6-yl-piperidin-1-yl-methanone), CX-717, CX-701, CX-1739, CX-1763, and CX-1837; benzothiazide derivatives such as cyclothiazide and IDRA-21 (7-chloro-3-methyl-3,4-dihydro-2H-1,2,4-benzothiadiazine 1,1-dioxide); biarylpropylsulfonamides such as LY-392,098, LY-404,187 (N-[2-(4′-cyanobiphenyl-4-yl)propyl]propane-2-sulfonamide), LY-451,646 and LY-503,430 (4′-{(1S)-1-fluoro-2-[(isopropylsulfonyl)amino]-1-methylethyl}-N-methylbiphenyl-4-carboxamide).

A synergistic effect may be calculated, for example, using suitable methods such as, for example, the Sigmoid-Eₘₐₓ equation (Holford & Scheiner, 19981, Clin. Pharmacokinet. 6: 429-453), the equation of Loewe additivity (Loewe & Muischnek, 1926, Arch. Exp. Pathol Pharmacol. 114: 313-326) and the median-effect equation (Chou & Talalay, 1984, Adv. Enzyme Regul. 22: 27-55). Each equation referred to above may be applied to experimental data to generate a corresponding graph to aid in assessing the effects of the drug combination. The corresponding graphs associated with the equations referred to above are the concentration-effect curve, isobologram curve and combination index curve, respectively.

### Pharmaceutical Compositions and Formulations

The invention also encompasses the use of pharmaceutical compositions of the compound (+)-doxapram, deuterated derivative thereof or a salt thereof to practice the methods of the invention, wherein the compositions are essentially free of (−)-doxapram, deuterated derivative thereof or a salt thereof.

Such a pharmaceutical composition may consist of the compound (+)-doxapram, deuterated derivative thereof or a salt thereof alone, wherein the compositions is essentially free of (−)-doxapram, a deuterated derivative thereof or a salt thereof, in a form suitable for administration to a subject, or the pharmaceutical composition may comprise the compound (+)-doxapram, a deuterated derivative thereof or a salt thereof, wherein the compositions is essentially free of (−)-doxapram, a deuterated derivative thereof or a salt thereof, and one or more pharmaceutically acceptable carriers, one or more additional ingredients, or some combination of these. The compound (+)-doxapram, or deuterated derivative thereof may be present in the pharmaceutical composition in the form of a physiologically acceptable salt, such as in combination with a physiologically acceptable anion, as is well known in the art.

In an embodiment, the pharmaceutical compositions useful for practicing the method of the invention may be administered to deliver a dose of between 1 ng/kg/day and 100 mg/kg/day. In another embodiment, the pharmaceutical compositions useful for practicing the invention may be administered to deliver a dose of between 1 ng/kg/day and 500 mg/kg/day.

The relative amounts of the active ingredient, the pharmaceutically acceptable carrier, and any additional ingredients in a pharmaceutical composition of the invention will vary, depending upon the identity, size, and condition of the subject treated and further depending upon the route by which the composition is to be administered. By way of example, the composition may comprise between 0.1% and 100% (w/w) active ingredient.

Pharmaceutical compositions that are useful in the methods of the invention may be suitably developed for inhalational, oral, rectal, vaginal, parenteral, topical, transdermal, pulmonary, intranasal, buccal, ophthalmic, intrathecal, intravenous or another route of administration. A composition useful within the methods of the invention may be directly administered to the brain, the brainstem, or any other part of the central nervous system of a mammal. Other contemplated formulations include projected nanoparticles, liposomal preparations, resealed erythrocytes containing the active ingredient, and immunologically-based formulations. The route(s) of administration will be readily apparent to the skilled artisan and will depend upon any number of factors including the type and severity of the disease being treated, the type and age of the veterinary or human patient being treated, and the like.

The formulations of the pharmaceutical compositions described herein may be prepared by any method known or hereafter developed in the art of pharmacology. In general, such preparatory methods include the step of bringing the active ingredient into association with a carrier or one or more other accessory ingredients, and then, if necessary or desirable, shaping or packaging the product into a desired single- or multi-dose unit.

As used herein, a “unit dose” is a discrete amount of the pharmaceutical composition comprising a predetermined amount of the active ingredient. The amount of the active ingredient is generally equal to the dosage of the active ingredient that would be administered to a subject or a convenient fraction of such a dosage such as, for example, one-half or one-third of such a dosage. The unit dosage form may be for a single daily dose or one of multiple daily doses (e.g., about 1 to 4 or more times per day). When multiple daily doses are used, the unit dosage form may be the same or different for each dose.

Although the descriptions of pharmaceutical compositions provided herein are principally directed to pharmaceutical compositions which are suitable for ethical administration to humans, it will be understood by the skilled artisan that such compositions are generally suitable for administration to animals of all sorts. Modification of pharmaceutical compositions suitable for administration to humans in order to render the compositions suitable for administration to various animals is well understood, and the ordinarily skilled veterinary pharmacologist can design and perform such modification with merely ordinary, if any, experimentation. Subjects to which administration of the pharmaceutical compositions of the invention is contemplated include, but are not limited to, humans and other primates, mammals including commercially relevant mammals such as cattle, pigs, horses, sheep, cats, and dogs.

In one embodiment, the compositions of the invention are formulated using one or more pharmaceutically acceptable excipients or carriers. In one embodiment, the pharmaceutical compositions of the invention comprise a therapeutically effective amount of a compound of the invention and a pharmaceutically acceptable carrier. Pharmaceutically acceptable carriers, which are useful, include, but are not limited to, glycerol, water, saline, ethanol and other pharmaceutically acceptable salt solutions such as phosphates and salts of organic acids. Examples of these and other pharmaceutically acceptable carriers are described in Remington's Pharmaceutical Sciences (1991, Mack Publication Co., New Jersey).

The carrier may be a solvent or dispersion medium containing, for example, water, ethanol, polyol (for example, glycerol, propylene glycol, and liquid polyethylene glycol, and the like), suitable mixtures thereof, and vegetable oils. The proper fluidity may be maintained, for example, by the use of a coating such as lecithin, by the maintenance of the required particle size in the case of dispersion and by the use of surfactants. Prevention of the action of microorganisms may be achieved by various antibacterial and antifungal agents, for example, parabens, chlorobutanol, phenol, ascorbic acid, thimerosal, and the like. In many cases, it will be preferable to include isotonic agents, for example, sugars, sodium chloride, or polyalcohols such as mannitol and sorbitol, in the composition. Prolonged absorption of the injectable compositions may be brought about by including in the composition an agent which delays absorption, for example, aluminum monostearate or gelatin. In one embodiment, the pharmaceutically acceptable carrier is not DMSO alone.

Formulations may be employed in admixtures with conventional excipients, i.e., pharmaceutically acceptable organic or inorganic carrier substances suitable for oral, parenteral, nasal, intravenous, subcutaneous, enteral, or any other suitable mode of administration, known to the art. The pharmaceutical preparations may be sterilized and if desired mixed with auxiliary agents, e.g., lubricants, preservatives, stabilizers, wetting agents, emulsifiers, salts for influencing osmotic pressure buffers, coloring, flavoring and/or aromatic substances and the like. They may also be combined where desired with other active agents, e.g., other analgesic agents.

As used herein, “additional ingredients” include, but are not limited to, one or more of the following: excipients; surface active agents; dispersing agents; inert diluents; granulating and disintegrating agents; binding agents; lubricating agents; sweetening agents; flavoring agents; coloring agents; preservatives; physiologically degradable compositions such as gelatin; aqueous vehicles and solvents; oily vehicles and solvents; suspending agents; dispersing or wetting agents; emulsifying agents, demulcents; buffers; salts; thickening agents; fillers; emulsifying agents; antioxidants; antibiotics; antifungal agents; stabilizing agents; and pharmaceutically acceptable polymeric or hydrophobic materials. Other “additional ingredients” which may be included in the pharmaceutical compositions of the invention are known in the art and described, for example in Genaro, ed. (1985, Remington's Pharmaceutical Sciences, Mack Publishing Co., Easton, Pa.), which is incorporated herein by reference.

The composition of the invention may comprise a preservative from about 0.005% to 2.0% by total weight of the composition. The preservative is used to prevent spoilage in the case of exposure to contaminants in the environment. Examples of preservatives useful in accordance with the invention included but are not limited to those selected from the group consisting of benzyl alcohol, sorbic acid, parabens, imidurea and combinations thereof. A particularly preferred preservative is a combination of about 0.5% to 2.0% benzyl alcohol and 0.05% to 0.5% sorbic acid.

The composition preferably includes an antioxidant and a chelating agent which inhibit the degradation of the compound. Preferred antioxidants for some compounds are BHT, BHA, alpha-tocopherol and ascorbic acid in the preferred range of about 0.01% to 0.3% and more preferably BHT in the range of 0.03% to 0.1% by weight by total weight of the composition. Preferably, the chelating agent is present in an amount of from 0.01% to 0.5% by weight by total weight of the composition. Particularly preferred chelating agents include edetate salts (e.g. disodium edetate) and citric acid in the weight range of about 0.01% to 0.20% and more preferably in the range of 0.02% to 0.10% by weight by total weight of the composition. The chelating agent is useful for chelating metal ions in the composition which may be detrimental to the shelf life of the formulation. While BHT and disodium edetate are the particularly preferred antioxidant and chelating agent respectively for some compounds, other suitable and equivalent antioxidants and chelating agents may be substituted therefore as would be known to those skilled in the art.

Liquid suspensions may be prepared using conventional methods to achieve suspension of the active ingredient in an aqueous or oily vehicle. Aqueous vehicles include, for example, water, and isotonic saline. Oily vehicles include, for example, almond oil, oily esters, ethyl alcohol, vegetable oils such as arachis, olive, sesame, or coconut oil, fractionated vegetable oils, and mineral oils such as liquid paraffin. Liquid suspensions may further comprise one or more additional ingredients including, but not limited to, suspending agents, dispersing or wetting agents, emulsifying agents, demulcents, preservatives, buffers, salts, flavorings, coloring agents, and sweetening agents. Oily suspensions may further comprise a thickening agent. Known suspending agents include, but are not limited to, sorbitol syrup, hydrogenated edible fats, sodium alginate, polyvinylpyrrolidone, gum tragacanth, gum acacia, and cellulose derivatives such as sodium carboxymethylcellulose, methylcellulose, hydroxypropylmethylcellulose. Known dispersing or wetting agents include, but are not limited to, naturally-occurring phosphatides such as lecithin, condensation products of an alkylene oxide with a fatty acid, with a long chain aliphatic alcohol, with a partial ester derived from a fatty acid and a hexitol, or with a partial ester derived from a fatty acid and a hexitol anhydride (e.g., polyoxyethylene stearate, heptadecaethyleneoxycetanol, polyoxyethylene sorbitol monooleate, and polyoxyethylene sorbitan monooleate, respectively). Known emulsifying agents include, but are not limited to, lecithin, and acacia. Known preservatives include, but are not limited to, methyl, ethyl, or n-propyl para-hydroxybenzoates, ascorbic acid, and sorbic acid. Known sweetening agents include, for example, glycerol, propylene glycol, sorbitol, sucrose, and saccharin. Known thickening agents for oily suspensions include, for example, beeswax, hard paraffin, and cetyl alcohol.

Liquid solutions of the active ingredient in aqueous or oily solvents may be prepared in substantially the same manner as liquid suspensions, the primary difference being that the active ingredient is dissolved, rather than suspended in the solvent. As used herein, an “oily” liquid is one which comprises a carbon-containing liquid molecule and which exhibits a less polar character than water. Liquid solutions of the pharmaceutical composition of the invention may comprise each of the components described with regard to liquid suspensions, it being understood that suspending agents will not necessarily aid dissolution of the active ingredient in the solvent. Aqueous solvents include, for example, water, and isotonic saline. Oily solvents include, for example, almond oil, oily esters, ethyl alcohol, vegetable oils such as arachis, olive, sesame, or coconut oil, fractionated vegetable oils, and mineral oils such as liquid paraffin.

Powdered and granular formulations of a pharmaceutical preparation of the invention may be prepared using known methods. Such formulations may be administered directly to a subject, used, for example, to form tablets, to fill capsules, or to prepare an aqueous or oily suspension or solution by addition of an aqueous or oily vehicle thereto. Each of these formulations may further comprise one or more of dispersing or wetting agent, a suspending agent, and a preservative. Additional excipients, such as fillers and sweetening, flavoring, or coloring agents, may also be included in these formulations.

A pharmaceutical composition of the invention may also be prepared, packaged, or sold in the form of oil-in-water emulsion or a water-in-oil emulsion. The oily phase may be a vegetable oil such as olive or arachis oil, a mineral oil such as liquid paraffin, or a combination of these. Such compositions may further comprise one or more emulsifying agents such as naturally occurring gums such as gum acacia or gum tragacanth, naturally-occurring phosphatides such as soybean or lecithin phosphatide, esters or partial esters derived from combinations of fatty acids and hexitol anhydrides such as sorbitan monooleate, and condensation products of such partial esters with ethylene oxide such as polyoxyethylene sorbitan monooleate. These emulsions may also contain additional ingredients including, for example, sweetening or flavoring agents.

Methods for impregnating or coating a material with a chemical composition are known in the art, and include, but are not limited to methods of depositing or binding a chemical composition onto a surface, methods of incorporating a chemical composition into the structure of a material during the synthesis of the material (i.e., such as with a physiologically degradable material), and methods of absorbing an aqueous or oily solution or suspension into an absorbent material, with or without subsequent drying.

### Administration/Dosing

The regimen of administration may affect what constitutes an effective amount. The therapeutic formulations may be administered to the patient either prior to or after the onset of a breathing control disorder event. Further, several divided dosages, as well as staggered dosages may be administered daily or sequentially, or the dose may be continuously infused, or may be a bolus injection. Further, the dosages of the therapeutic formulations may be proportionally increased or decreased as indicated by the exigencies of the therapeutic or prophylactic situation.

Administration of the compositions of the present invention to a patient, preferably a mammal, more preferably a human, may be carried out using known procedures, at dosages and for periods of time effective to treat a breathing control disorder in the patient. An effective amount of the therapeutic compound necessary to achieve a therapeutic effect may vary according to factors such as the activity of the particular compound employed; the time of administration; the rate of excretion of the compound; the duration of the treatment; other drugs, compounds or materials used in combination with the compound; the state of the disease or disorder, age, sex, weight, condition, general health and prior medical history of the patient being treated, and like factors well-known in the medical arts. Dosage regimens may be adjusted to provide the optimum therapeutic response. For example, several divided doses may be administered daily or the dose may be proportionally reduced as indicated by the exigencies of the therapeutic situation. A non-limiting example of an effective dose range for a therapeutic compound of the invention is from about 0.01 and 50 mg/kg of body weight/per day. One of ordinary skill in the art would be able to study the relevant factors and make the determination regarding the effective amount of the therapeutic compound without undue experimentation.

The compound can be administered to an animal as frequently as several times daily, or it may be administered less frequently, such as once a day, once a week, once every two weeks, once a month, or even less frequently, such as once every several months or even once a year or less. It is understood that the amount of compound dosed per day may be administered, in non-limiting examples, every day, every other day, every 2 days, every 3 days, every 4 days, or every 5 days. For example, with every other day administration, a 5 mg per day dose may be initiated on Monday with a first subsequent 5 mg per day dose administered on Wednesday, a second subsequent 5 mg per day dose administered on Friday, and so on. The frequency of the dose will be readily apparent to the skilled artisan and will depend upon any number of factors, such as, but not limited to, the type and severity of the disease being treated, the type and age of the animal, etc.

Actual dosage levels of the active ingredients in the pharmaceutical compositions of this invention may be varied so as to obtain an amount of the active ingredient that is effective to achieve the desired therapeutic response for a particular patient, composition, and mode of administration, without being toxic to the patient.

A medical doctor, e.g., physician or veterinarian, having ordinary skill in the art may readily determine and prescribe the effective amount of the pharmaceutical composition required. For example, the physician or veterinarian could start doses of the compounds of the invention employed in the pharmaceutical composition at levels lower than that required in order to achieve the desired therapeutic effect and gradually increase the dosage until the desired effect is achieved.

In particular embodiments, it is especially advantageous to formulate the compound in dosage unit form for ease of administration and uniformity of dosage. Dosage unit form as used herein refers to physically discrete units suited as unitary dosages for the patients to be treated; each unit containing a predetermined quantity of therapeutic compound calculated to produce the desired therapeutic effect in association with the required pharmaceutical vehicle. The dosage unit forms of the invention are dictated by and directly dependent on (a) the unique characteristics of the therapeutic compound and the particular therapeutic effect to be achieved, and (b) the limitations inherent in the art of compounding/formulating such a therapeutic compound for the treatment of breathing control disorders in a patient.

In one embodiment, the compositions of the invention are administered to the patient in dosages that range from one to five times per day or more. In another embodiment, the compositions of the invention are administered to the patient in range of dosages that include, but are not limited to, once every day, every two, days, every three days to once a week, and once every two weeks. It will be readily apparent to one skilled in the art that the frequency of administration of the various combination compositions of the invention will vary from subject to subject depending on many factors including, but not limited to, age, disease or disorder to be treated, gender, overall health, and other factors. Thus, the invention should not be construed to be limited to any particular dosage regime and the precise dosage and composition to be administered to any patient will be determined by the attending physical taking all other factors about the patient into account.

Compounds of the invention for administration may be in the range of from about 1 μg to about 7,500 mg, about 20 μg to about 7,000 mg, about 40 μg to about 6,500 mg, about 80 μg to about 6,000 mg, about 100 μg to about 5,500 mg, about 200 μg to about 5,000 mg, about 400 μg to about 4,000 mg, about 800 μg to about 3,000 mg, about 1 mg to about 2,500 mg, about 2 mg to about 2,000 mg, about 5 mg to about 1,000 mg, about 10 mg to about 750 mg, about 20 mg to about 600 mg, about 30 mg to about 500 mg, about 40 mg to about 400 mg, about 50 mg to about 300 mg, about 60 mg to about 250 mg, about 70 mg to about 200 mg, about 80 mg to about 150 mg, and any and all whole or partial increments therebetween.

In some embodiments, the dose of a compound of the invention is from about 0.5 μg and about 5,000 mg. In some embodiments, a dose of a compound of the invention used in compositions described herein is less than about 5,000 mg, or less than about 4,000 mg, or less than about 3,000 mg, or less than about 2,000 mg, or less than about 1,000 mg, or less than about 800 mg, or less than about 600 mg, or less than about 500 mg, or less than about 200 mg, or less than about 50 mg. Similarly, in some embodiments, a dose of a second compound as described herein is less than about 1,000 mg, or less than about 800 mg, or less than about 600 mg, or less than about 500 mg, or less than about 400 mg, or less than about 300 mg, or less than about 200 mg, or less than about 100 mg, or less than about 50 mg, or less than about 40 mg, or less than about 30 mg, or less than about 25 mg, or less than about 20 mg, or less than about 15 mg, or less than about 10 mg, or less than about 5 mg, or less than about 2 mg, or less than about 1 mg, or less than about 0.5 mg, and any and all whole or partial increments thereof.

In one embodiment, the present invention is directed to a packaged pharmaceutical composition comprising a container holding a therapeutically effective amount of a compound of the invention, alone or in combination with a second pharmaceutical agent; and instructions for using the compound to treat, prevent, or reduce one or more symptoms of breathing control disorder in a patient.

The term “container” includes any receptacle for holding the pharmaceutical composition. For example, in one embodiment, the container is the packaging that contains the pharmaceutical composition. In other embodiments, the container is not the packaging that contains the pharmaceutical composition, i.e., the container is a receptacle, such as a box or vial that contains the packaged pharmaceutical composition or unpackaged pharmaceutical composition and the instructions for use of the pharmaceutical composition. Moreover, packaging techniques are well known in the art. It should be understood that the instructions for use of the pharmaceutical composition may be contained on the packaging containing the pharmaceutical composition, and as such the instructions form an increased functional relationship to the packaged product. However, it should be understood that the instructions may contain information pertaining to the compound's ability to perform its intended function, e.g., treating, preventing, or reducing a breathing control disorder in a patient.

### Routes of Administration

Routes of administration of any of the compositions of the invention include inhalational, oral, nasal, rectal, parenteral, sublingual, transdermal, transmucosal (e.g., sublingual, lingual, (trans)buccal, (trans)urethral, vaginal (e.g., trans- and perivaginally), (intra)nasal, and (trans)rectal, intravesical, intrapulmonary, intraduodenal, intragastrical, intrathecal, subcutaneous, intramuscular, intradermal, intra-arterial, intravenous, intrabronchial, inhalation, and topical administration.

Suitable compositions and dosage forms include, for example, tablets, capsules, caplets, pills, gel caps, troches, dispersions, suspensions, solutions, syrups, granules, beads, transdermal patches, gels, powders, pellets, magmas, lozenges, creams, pastes, plasters, lotions, discs, suppositories, liquid sprays for nasal or oral administration, dry powder or aerosolized formulations for inhalation, compositions and formulations for intravesical administration and the like. It should be understood that the formulations and compositions that would be useful in the present invention are not limited to the particular formulations and compositions that are described herein.

### Oral Administration

For oral application, particularly suitable are tablets, dragees, liquids, drops, suppositories, or capsules, caplets and gelcaps. Other formulations suitable for oral administration include, but are not limited to, a powdered or granular formulation, an aqueous or oily suspension, an aqueous or oily solution, a paste, a gel, toothpaste, a mouthwash, a coating, an oral rinse, or an emulsion. The compositions intended for oral use may be prepared according to any method known in the art and such compositions may contain one or more agents selected from the group consisting of inert, non-toxic pharmaceutically excipients which are suitable for the manufacture of tablets. Such excipients include, for example an inert diluent such as lactose; granulating and disintegrating agents such as cornstarch; binding agents such as starch; and lubricating agents such as magnesium stearate.

Tablets may be non-coated or they may be coated using known methods to achieve delayed disintegration in the gastrointestinal tract of a subject, thereby providing sustained release and absorption of the active ingredient. By way of example, a material such as glyceryl monostearate or glyceryl distearate may be used to coat tablets. Further by way of example, tablets may be coated using methods described in U.S. Pat. Nos. 4,256,108; 4,160,452; and 4,265,874 to form osmotically controlled release tablets. Tablets may further comprise a sweetening agent, a flavoring agent, a coloring agent, a preservative, or some combination of these in order to provide for pharmaceutically elegant and palatable preparation.

Hard capsules comprising the active ingredient may be made using a physiologically degradable composition, such as gelatin. Such hard capsules comprise the active ingredient, and may further comprise additional ingredients including, for example, an inert solid diluent such as calcium carbonate, calcium phosphate, or kaolin.

Soft gelatin capsules comprising the active ingredient may be made using a physiologically degradable composition, such as gelatin. Such soft capsules comprise the active ingredient, which may be mixed with water or an oil medium such as peanut oil, liquid paraffin, or olive oil.

For oral administration, the compounds of the invention may be in the form of tablets or capsules prepared by conventional means with pharmaceutically acceptable excipients such as binding agents; fillers; lubricants; disintegrates; or wetting agents. If desired, the tablets may be coated using suitable methods and coating materials such as OPADRY™ film coating systems available from Colorcon, West Point, Pa. (e.g., OPADRY™ OY Type, OYC Type, Organic Enteric OY-P Type, Aqueous Enteric OY-A Type, OY-PM Type and OPADRY™ White, 321(18400).

Liquid preparation for oral administration may be in the form of solutions, syrups or suspensions. The liquid preparations may be prepared by conventional means with pharmaceutically acceptable additives such as suspending agents (e.g., sorbitol syrup, methyl cellulose or hydrogenated edible fats); emulsifying agent (e.g., lecithin or acacia); non-aqueous vehicles (e.g., almond oil, oily esters or ethyl alcohol); and preservatives (e.g., methyl or propyl para-hydroxy benzoates or sorbic acid). Liquid formulations of a pharmaceutical composition of the invention which are suitable for oral administration may be prepared, packaged, and sold either in liquid form or in the form of a dry product intended for reconstitution with water or another suitable vehicle prior to use.

A tablet comprising the active ingredient may, for example, be made by compressing or molding the active ingredient, optionally with one or more additional ingredients. Compressed tablets may be prepared by compressing, in a suitable device, the active ingredient in a free-flowing form such as a powder or granular preparation, optionally mixed with one or more of a binder, a lubricant, an excipient, a surface active agent, and a dispersing agent. Molded tablets may be made by molding, in a suitable device, a mixture of the active ingredient, a pharmaceutically acceptable carrier, and at least sufficient liquid to moisten the mixture. Pharmaceutically acceptable excipients used in the manufacture of tablets include, but are not limited to, inert diluents, granulating and disintegrating agents, binding agents, and lubricating agents. Known dispersing agents include, but are not limited to, potato starch and sodium starch glycollate. Known surface-active agents include, but are not limited to, sodium lauryl sulphate. Known diluents include, but are not limited to, calcium carbonate, sodium carbonate, lactose, microcrystalline cellulose, calcium phosphate, calcium hydrogen phosphate, and sodium phosphate. Known granulating and disintegrating agents include, but are not limited to, corn starch and alginic acid. Known binding agents include, but are not limited to, gelatin, acacia, pre-gelatinized maize starch, polyvinylpyrrolidone, and hydroxypropyl methylcellulose. Known lubricating agents include, but are not limited to, magnesium stearate, stearic acid, silica, and talc.

Granulating techniques are well known in the pharmaceutical art for modifying starting powders or other particulate materials of an active ingredient. The powders are typically mixed with a binder material into larger permanent free-flowing agglomerates or granules referred to as a “granulation.” For example, solvent-using “wet” granulation processes are generally characterized in that the powders are combined with a binder material and moistened with water or an organic solvent under conditions resulting in the formation of a wet granulated mass from which the solvent must then be evaporated.

Melt granulation generally consists in the use of materials that are solid or semi-solid at room temperature (i.e. having a relatively low softening or melting point range) to promote granulation of powdered or other materials, essentially in the absence of added water or other liquid solvents. The low melting solids, when heated to a temperature in the melting point range, liquefy to act as a binder or granulating medium. The liquefied solid spreads itself over the surface of powdered materials with which it is contacted, and on cooling, forms a solid granulated mass in which the initial materials are bound together. The resulting melt granulation may then be provided to a tablet press or be encapsulated for preparing the oral dosage form. Melt granulation improves the dissolution rate and bioavailability of an active (i.e. drug) by forming a solid dispersion or solid solution.

U.S. Pat. No. 5,169,645 discloses directly compressible wax-containing granules having improved flow properties. The granules are obtained when waxes are admixed in the melt with certain flow improving additives, followed by cooling and granulation of the admixture. In certain embodiments, only the wax itself melts in the melt combination of the wax(es) and additives(s), and in other cases both the wax(es) and the additives(s) will melt.

The present invention also includes a multi-layer tablet comprising a layer providing for the delayed release of one or more compounds useful within the methods of the invention, and a further layer providing for the immediate release of one or more compounds useful within the methods of the invention. Using a wax/pH-sensitive polymer mix, a gastric insoluble composition may be obtained in which the active ingredient is entrapped, ensuring its delayed release.

### Parenteral Administration

As used herein, “parenteral administration” of a pharmaceutical composition includes any route of administration characterized by physical breaching of a tissue of a subject and administration of the pharmaceutical composition through the breach in the tissue. Parenteral administration thus includes, but is not limited to, administration of a pharmaceutical composition by injection of the composition, by application of the composition through a surgical incision, by application of the composition through a tissue-penetrating non-surgical wound, and the like. In particular, parenteral administration is contemplated to include, but is not limited to, subcutaneous, intravenous, intraperitoneal, intramuscular, intrasternal injection, and kidney dialytic infusion techniques.

Formulations of a pharmaceutical composition suitable for parenteral administration comprise the active ingredient combined with a pharmaceutically acceptable carrier, such as sterile water or sterile isotonic saline. Such formulations may be prepared, packaged, or sold in a form suitable for bolus administration or for continuous administration. Injectable formulations may be prepared, packaged, or sold in unit dosage form, such as in ampules or in multi-dose containers containing a preservative. Formulations for parenteral administration include, but are not limited to, suspensions, solutions, emulsions in oily or aqueous vehicles, pastes, and implantable sustained-release or biodegradable formulations. Such formulations may further comprise one or more additional ingredients including, but not limited to, suspending, stabilizing, or dispersing agents. In one embodiment of a formulation for parenteral administration, the active ingredient is provided in dry (i.e., powder or granular) form for reconstitution with a suitable vehicle (e.g., sterile pyrogen-free water) prior to parenteral administration of the reconstituted composition.

The pharmaceutical compositions may be prepared, packaged, or sold in the form of a sterile injectable aqueous or oily suspension or solution. This suspension or solution may be formulated according to the known art, and may comprise, in addition to the active ingredient, additional ingredients such as the dispersing agents, wetting agents, or suspending agents described herein. Such sterile injectable formulations may be prepared using a non-toxic parenterally-acceptable diluent or solvent, such as water or 1,3-butane diol, for example. Other acceptable diluents and solvents include, but are not limited to, Ringer's solution, isotonic sodium chloride solution, and fixed oils such as synthetic mono- or di-glycerides. Other parentally-administrable formulations which are useful include those which comprise the active ingredient in microcrystalline form, in a liposomal preparation, or as a component of a biodegradable polymer system. Compositions for sustained release or implantation may comprise pharmaceutically acceptable polymeric or hydrophobic materials such as an emulsion, an ion exchange resin, a sparingly soluble polymer, or a sparingly soluble salt.

### Topical Administration

An obstacle for topical administration of pharmaceuticals is the stratum corneum layer of the epidermis. The stratum corneum is a highly resistant layer comprised of protein, cholesterol, sphingolipids, free fatty acids and various other lipids, and includes cornified and living cells. One of the factors that limit the penetration rate (flux) of a compound through the stratum corneum is the amount of the active substance that can be loaded or applied onto the skin surface. The greater the amount of active substance which is applied per unit of area of the skin, the greater the concentration gradient between the skin surface and the lower layers of the skin, and in turn the greater the diffusion force of the active substance through the skin. Therefore, a formulation containing a greater concentration of the active substance is more likely to result in penetration of the active substance through the skin, and more of it, and at a more consistent rate, than a formulation having a lesser concentration, all other things being equal.

Formulations suitable for topical administration include, but are not limited to, liquid or semi-liquid preparations such as liniments, lotions, oil-in-water or water-in-oil emulsions such as creams, ointments or pastes, and solutions or suspensions. Topically administrable formulations may, for example, comprise from about 1% to about 10% (w/w) active ingredient, although the concentration of the active ingredient may be as high as the solubility limit of the active ingredient in the solvent. Formulations for topical administration may further comprise one or more of the additional ingredients described herein.

Enhancers of permeation may be used. These materials increase the rate of penetration of drugs across the skin. Typical enhancers in the art include ethanol, glycerol monolaurate, PGML (polyethylene glycol monolaurate), dimethylsulfoxide, and the like. Other enhancers include oleic acid, oleyl alcohol, ethoxydiglycol, laurocapram, alkanecarboxylic acids, dimethylsulfoxide, polar lipids, or N-methyl-2-pyrrolidone.

One acceptable vehicle for topical delivery of some of the compositions of the invention may contain liposomes. The composition of the liposomes and their use are known in the art (for example, see Constanza, U.S. Pat. No. 6,323,219).

In alternative embodiments, the topically active pharmaceutical composition may be optionally combined with other ingredients such as adjuvants, anti- oxidants, chelating agents, surfactants, foaming agents, wetting agents, emulsifying agents, viscosifiers, buffering agents, preservatives, and the like. In another embodiment, a permeation or penetration enhancer is included in the composition and is effective in improving the percutaneous penetration of the active ingredient into and through the stratum corneum with respect to a composition lacking the permeation enhancer. Various permeation enhancers, including oleic acid, oleyl alcohol, ethoxydiglycol, laurocapram, alkanecarboxylic acids, dimethylsulfoxide, polar lipids, or N-methyl-2-pyrrolidone, are known to those of skill in the art. In another aspect, the composition may further comprise a hydrotropic agent, which functions to increase disorder in the structure of the stratum corneum, and thus allows increased transport across the stratum corneum. Various hydrotropic agents such as isopropyl alcohol, propylene glycol, or sodium xylene sulfonate, are known to those of skill in the art.

The topically active pharmaceutical composition should be applied in an amount effective to affect desired changes. As used herein “amount effective” shall mean an amount sufficient to cover the region of skin surface where a change is desired. An active compound should be present in the amount of from about 0.0001% to about 15% by weight volume of the composition. More preferable, it should be present in an amount from about 0.0005% to about 5% of the composition; most preferably, it should be present in an amount of from about 0.001% to about 1% of the composition. Such compounds may be synthetically-or naturally derived.

### Buccal Administration

A pharmaceutical composition of the invention may be prepared, packaged, or sold in a formulation suitable for buccal administration. Such formulations may, for example, be in the form of tablets or lozenges made using conventional methods, and may contain, for example, 0.1 to 20% (w/w) of the active ingredient, the balance comprising an orally dissolvable or degradable composition and, optionally, one or more of the additional ingredients described herein. Alternately, formulations suitable for buccal administration may comprise a powder or an aerosolized or atomized solution or suspension comprising the active ingredient. Such powdered, aerosolized, or aerosolized formulations, when dispersed, preferably have an average particle or droplet size in the range from about 0.1 to about 200 nanometers, and may further comprise one or more of the additional ingredients described herein. The examples of formulations described herein are not exhaustive and it is understood that the invention includes additional modifications of these and other formulations not described herein, but which are known to those of skill in the art.

### Rectal Administration

A pharmaceutical composition of the invention may be prepared, packaged, or sold in a formulation suitable for rectal administration. Such a composition may be in the form of, for example, a suppository, a retention enema preparation, and a solution for rectal or colonic irrigation.

Suppository formulations may be made by combining the active ingredient with a non-irritating pharmaceutically acceptable excipient which is solid at ordinary room temperature (i.e., about 20° C.) and which is liquid at the rectal temperature of the subject (i.e., about 37° C. in a healthy human). Suitable pharmaceutically acceptable excipients include, but are not limited to, cocoa butter, polyethylene glycols, and various glycerides. Suppository formulations may further comprise various additional ingredients including, but not limited to, antioxidants, and preservatives.

Retention enema preparations or solutions for rectal or colonic irrigation may be made by combining the active ingredient with a pharmaceutically acceptable liquid carrier. As is well known in the art, enema preparations may be administered using, and may be packaged within, a delivery device adapted to the rectal anatomy of the subject. Enema preparations may further comprise various additional ingredients including, but not limited to, antioxidants, and preservatives.

### Additional Administration Forms

Additional dosage forms of this invention include dosage forms as described in U.S. Pat. Nos. 6,340,475, 6,488,962, 6,451,808, 5,972,389, 5,582,837, and 5,007,790. Additional dosage forms of this invention also include dosage forms as described in U.S. Patent Applications Nos. 20030147952, 20030104062, 20030104053, 20030044466, 20030039688, and 20020051820. Additional dosage forms of this invention also include dosage forms as described in PCT Applications Nos. WO 03/35041, WO 03/35040, WO 03/35029, WO 03/35177, WO 03/35039, WO 02/96404, WO 02/32416, WO 01/97783, WO 01/56544, WO 01/32217, WO 98/55107, WO 98/11879, WO 97/47285, WO 93/18755, and WO 90/11757.

### Controlled Release Formulations and Drug Delivery Systems

Controlled- or sustained-release formulations of a pharmaceutical composition of the invention may be made using conventional technology. In some cases, the dosage forms to be used can be provided as slow or controlled-release of one or more active ingredients therein using, for example, hydropropylmethyl cellulose, other polymer matrices, gels, permeable membranes, osmotic systems, multilayer coatings, microparticles, liposomes, or microspheres or a combination thereof to provide the desired release profile in varying proportions. Suitable controlled-release formulations known to those of ordinary skill in the art, including those described herein, can be readily selected for use with the pharmaceutical compositions of the invention. Thus, single unit dosage forms suitable for oral administration, such as tablets, capsules, gelcaps, and caplets, that are adapted for controlled-release are encompassed by the present invention.

Most controlled-release pharmaceutical products have a common goal of improving drug therapy over that achieved by their non-controlled counterparts. Ideally, the use of an optimally designed controlled-release preparation in medical treatment is characterized by a minimum of drug substance being employed to cure or control the condition in a minimum amount of time. Advantages of controlled-release formulations include extended activity of the drug, reduced dosage frequency, and increased patient compliance. In addition, controlled-release formulations can be used to affect the time of onset of action or other characteristics, such as blood level of the drug, and thus can affect the occurrence of side effects.

Most controlled-release formulations are designed to initially release an amount of drug that promptly produces the desired therapeutic effect, and gradually and continually release of other amounts of drug to maintain this level of therapeutic effect over an extended period of time. In order to maintain this constant level of drug in the body, the drug must be released from the dosage form at a rate that will replace the amount of drug being metabolized and excreted from the body.

Controlled-release of an active ingredient can be stimulated by various inducers, for example pH, temperature, enzymes, water, or other physiological conditions or compounds. The term “controlled-release component” in the context of the present invention is defined herein as a compound or compounds, including, but not limited to, polymers, polymer matrices, gels, permeable membranes, liposomes, or microspheres or a combination thereof that facilitates the controlled-release of the active ingredient.

In certain embodiments, the formulations of the present invention may be, but are not limited to, short-term, rapid-offset, as well as controlled, for example, sustained release, delayed release and pulsatile release formulations.

The term sustained release is used in its conventional sense to refer to a drug formulation that provides for gradual release of a drug over an extended period of time, and that may, although not necessarily, result in substantially constant blood levels of a drug over an extended time period. The period of time may be as long as a month or more and should be a release which is longer that the same amount of agent administered in bolus form.

For sustained release, the compounds may be formulated with a suitable polymer or hydrophobic material which provides sustained release properties to the compounds. As such, the compounds for use the method of the invention may be administered in the form of microparticles, for example, by injection or in the form of wafers or discs by implantation.

In a preferred embodiment of the invention, the compounds of the invention are administered to a patient, alone or in combination with another pharmaceutical agent, using a sustained release formulation.

The term delayed release is used herein in its conventional sense to refer to a drug formulation that provides for an initial release of the drug after some delay following drug administration and that mat, although not necessarily, includes a delay of from about 10 minutes up to about 12 hours.

The term pulsatile release is used herein in its conventional sense to refer to a drug formulation that provides release of the drug in such a way as to produce pulsed plasma profiles of the drug after drug administration.

The term immediate release is used in its conventional sense to refer to a drug formulation that provides for release of the drug immediately after drug administration.

As used herein, short-term refers to any period of time up to and including about 8 hours, about 7 hours, about 6 hours, about 5 hours, about 4 hours, about 3 hours, about 2 hours, about 1 hour, about 40 minutes, about 20 minutes, or about 10 minutes and any or all whole or partial increments thereof after drug administration after drug administration.

As used herein, rapid-offset refers to any period of time up to and including about 8 hours, about 7 hours, about 6 hours, about 5 hours, about 4 hours, about 3 hours, about 2 hours, about 1 hour, about 40 minutes, about 20 minutes, or about 10 minutes, and any and all whole or partial increments thereof after drug administration.

### Mechanical Devices

In one aspect of the invention, a method of treating a patient lacking normal breathing control comprises administering the composition useful within the invention as described herein, and additionally treating the patient using a device for treatment of a lack of normal breathing control. Such devices include, but are not limited to, ventilation devices, CPAP and BiPAP devices.

Mechanical ventilation is a method to mechanically assist or replace spontaneous breathing. Mechanical ventilation is typically used after an invasive intubation, a procedure wherein an endotracheal or tracheostomy tube is inserted into the airway. It is normally used in acute settings, such as in the ICU, for a short period of time during a serious illness. It may also be used at home or in a nursing or rehabilitation institution, if patients have chronic illnesses that require long-term ventilation assistance. The main form of mechanical ventilation is positive pressure ventilation, which works by increasing the pressure in the patient's airway and thus forcing air into the lungs. Less common today are negative pressure ventilators (for example, the “iron lung”) that create a negative pressure environment around the patient's chest, thus sucking air into the lungs. Mechanical ventilation is often a life-saving intervention, but carries many potential complications including pneumothorax, airway injury, alveolar damage, and ventilator-associated pneumonia. For this reason the pressure and volume of gas used is strictly controlled, and reduced as soon as possible. Types of mechanical ventilation are: conventional ventilation, high frequency ventilation, non-invasive ventilation (non-invasive positive pressure pentilation or NIPPY), proportional assist ventilation (PAY), adaptive support ventilation (ASV) and neurally adjusted ventilatory assist (NAVA).

Non-invasive ventilation refers to all modalities that assist ventilation without the use of an endotracheal tube. Non-invasive ventilation is primarily aimed at minimizing patient discomfort and the complications associated with invasive ventilation, and is often used in cardiac disease, exacerbations of chronic pulmonary disease, sleep apnea, and neuromuscular diseases. Non-invasive ventilation refers only to the patient interface and not the mode of ventilation used; modes may include spontaneous or control modes and may be either pressure or volume modes. Some commonly used modes of NIPPY include:

(a) Continuous positive airway pressure (CPAP): This kind of machine has been used mainly by patients for the treatment of sleep apnea at home, but now is in widespread use across intensive care units as a form of ventilation. The CPAP machine stops upper airway obstruction by delivering a stream of compressed air via a hose to a nasal pillow, nose mask or full-face mask, stenting the airway (keeping it open under air pressure) so that unobstructed breathing becomes possible, reducing and/or preventing apneas and hypopneas. When the machine is turned on, but prior to the mask being placed on the head, a flow of air comes through the mask. After the mask is placed on the head, it is sealed to the face and the air stops flowing. At this point, it is only the air pressure that accomplishes the desired result. This has the additional benefit of reducing or eliminating the extremely loud snoring that sometimes accompanies sleep apnea.

(b) Bi-level positive airway pressure (BIPAP): Pressures alternate between inspiratory positive airway pressure (IPAP) and a lower expiratory positive airway pressure (EPAP), triggered by patient effort. On many such devices, backup rates may be set, which deliver IPAP pressures even if patients fail to initiate a breath.

(c) Intermittent positive pressure ventilation (IPPV), via mouthpiece or mask.

Those skilled in the art will recognize, or be able to ascertain using no more than routine experimentation, numerous equivalents to the specific procedures, embodiments, claims, and examples described herein. Such equivalents were considered to be within the scope of this invention and covered by the claims appended hereto. For example, it should be understood, that modifications in reaction conditions, including but not limited to reaction times, reaction size/volume, and experimental reagents, such as solvents, catalysts, pressures, atmospheric conditions, e.g., nitrogen atmosphere, and reducing/oxidizing agents, with art-recognized alternatives and using no more than routine experimentation, are within the scope of the present application.

It is to be understood that, wherever values and ranges are provided herein, the description in range format is merely for convenience and brevity and should not be construed as an inflexible limitation on the scope of the invention. Accordingly, all values and ranges encompassed by these values and ranges are meant to be encompassed within the scope of the present invention. Moreover, all values that fall within these ranges, as well as the upper or lower limits of a range of values, are also contemplated by the present application. The description of a range should be considered to have specifically disclosed all the possible sub-ranges as well as individual numerical values within that range and, when appropriate, partial integers of the numerical values within ranges. For example, description of a range such as from 1 to 6 should be considered to have specifically disclosed sub-ranges such as from 1 to 3, from 1 to 4, from 1 to 5, from 2 to 4, from 2 to 6, from 3 to 6 etc., as well as individual numbers within that range, for example, 1, 2, 2.7, 3, 4, 5, 5.3, and 6. This applies regardless of the breadth of the range.

The following examples further illustrate aspects of the present invention. However, they are in no way a limitation of the teachings or disclosure of the present invention as set forth herein.

### EXAMPLES

The invention is now described with reference to the following Examples. These Examples are provided for the purpose of illustration only, and the invention is not limited to these Examples, but rather encompasses all variations that are evident as a result of the teachings provided herein.

### Materials:

Unless otherwise noted, all remaining starting materials were obtained from commercial suppliers and used without purification. Unless otherwise noted, the vehicle used in the experiments was 15% DMA (dimethylacetamide): 85% PEG (polyethylene glycol).

### Preparative Example 1

### Chromatographic Separation of Racemic Doxapram

The operating conditions used for the separation process are as follows. The column was CHIRALPAK© AY 20 μm, 3 cm internal diameter×25 cm length. The mobile phase was EtOH with 0.2% DMEA (dimethylethylamine) and CO₂, in a ratio of 15:85. The flow rate was 85 g/min, the temperature of the column was kept at 35° C., and UV detection was performed at 220 nm.

The solubility of racemic doxapram was first determined to be 12.6 g/L in EtOH/MeOH (50/50 v/v). With stirring and sonication, 2.51 g of racemic doxapram were dissolved in approximately 200 mL EtOH/MeOH (50/50 v/v). The solution was injected onto the chromatographic column using the conditions illustrated above. The injection volume was 4 mL, performed every 7 minutes. The appropriate fractions collected from the chromatographic process were concentrated using rotary evaporators at 40° C. and 50 mbar. After solvent removal, the products were dried in a vacuum oven at 40° C. to obtain (−)-doxapram (1.19 g, 95% yield, ?99% e.e.) and (+)-doxapram (1.34 g, 107% yield, ≧99% e.e.). The experimental recovery of (+)-doxapram may have been high due to residual DMEA in the system.

TABLE 1

| Enantiomer | (−)-doxapram | (+)-doxapram |
|---|---|---|
| Weight (g) | 1.19 | 1.34 |
| % e.e. | >99.9 | 99.4 |
| Yield | 94.8% | 106.8% |

Optical rotation data was obtained with a flow injection using an Agilent 1200 HPLC and a PDR-Chiral Advanced Laser Polarimeter detector with methanol as mobile phase. Peak 1 by SFC is the (−)-enantiomer. Peak 2 by SFC is the (+)-enantiomer.

### Preparative Example 2

### Chromatographic Separation of Racemic Doxapram

The operating conditions used for the separation process are as follows. The column was CHIRALPAK® AY-H 5 μm, 3 cm internal diameter×25 cm length. The mobile phase was EtOH with 0.2% DMEA and CO₂, in a ratio of 15:85. The flow rate was 85 g/min. The temperature of the column was kept at 35° C., and UV absorption was monitored at 220 nm.

The solubility of racemic doxapram was determined to 19.9 g/L in EtOH/MeOH 80/20 (v/v). A sample of 19.94 g of racemic doxapram was dissolved in approximately 1.0 L of EtOH/MeOH 80/20 (v/v) with stirring and sonication. The injection volume was 4 mL, and injection was performed every 5.83 min. The appropriate fractions collected from the chromatographic process were concentrated using rotary evaporators at 40° C. and 50 mbar. After solvent removal, the products were dried in a vacuum oven at 40° C. to obtain (−)-doxapram (8.47 g, 85% yield, ≧99% e.e.) and (+)-doxapram (12.50 g, 125% yield, ≧99% e.e.). The experimental recovery of (+)-doxapram may have been high due to residual DMEA in the system.

TABLE 2

| Enantiomer | (−)-doxapram | (+)-doxapram |
|---|---|---|
| Weight (g) | 8.47 | 12.50 |
| % e.e. | >99.9 | 99.8 |
| Yield | 85.0% | 125.4% |

### Example 1

### Effect of (+)-Doxapram and (−)-Doxapram in Ventilation Parameters in the Rat, as Determined by In Vivo Spirometry

All surgical procedures were performed under anesthesia induced by 2% isoflurane in compressed medical grade air. With rats in supine position, the right femoral vein was catheterized using polyethylene tubing (PE-50). This catheter was used for fluid and drug administration. Simultaneously, the right femoral artery was also catheterized for monitoring blood pressure. In order to measure the respiratory parameters in spontaneously breathing rats, trachea was intubated using 13 gauge tracheal tube (2.5 mm ID, Instech Solomon, Pa.).

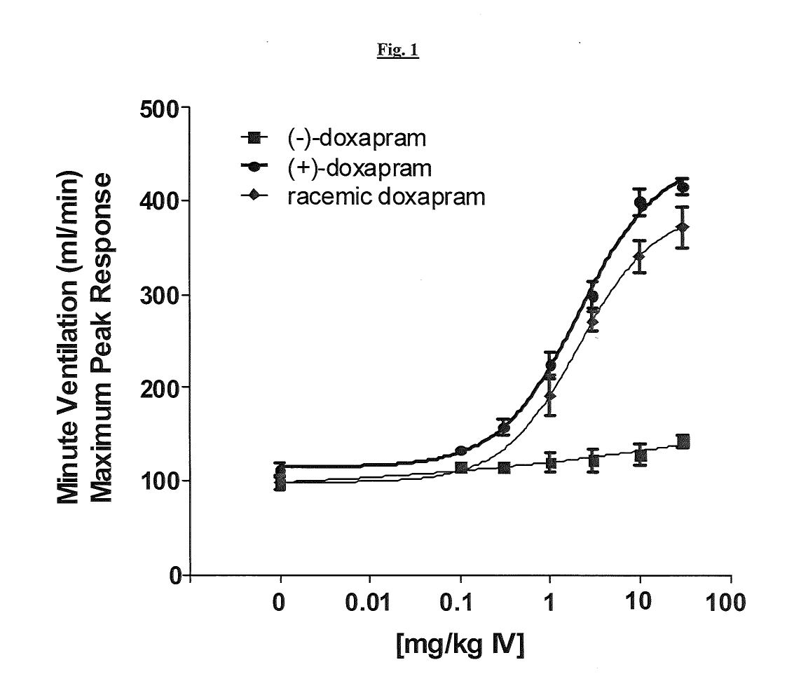

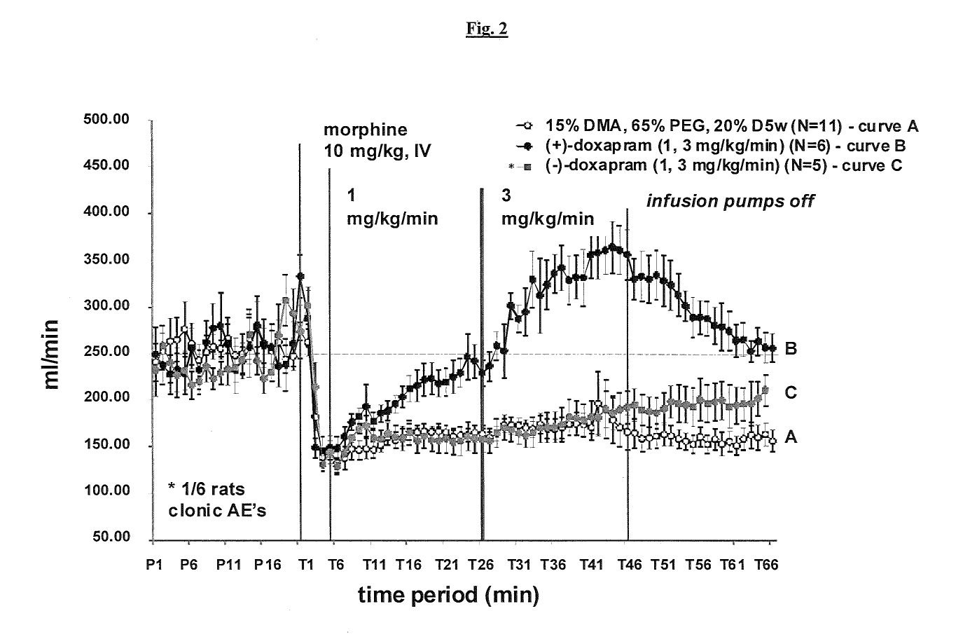

After establishing a stable base-line at 1.5% isoflurane, cumulative dose-dependent (1, 3, 10 and 30 mg/kg) ventilatory responses to (−)-doxapram, (+)-doxapram, or racemic doxapram were generated from spontaneously breathing rats. Maximum peak minute ventilatory (MV) values at each dose from corresponding drug were calculated and used for generating ED₅₀ values. Results are illustrated in Table 3 and FIG. 1. All the specific ventilatory stimulant activity of racemic doxapram was associated with the (+)-enantiomer. The (−)-enantiomer was at least 100-times less potent than the (+)-enantiomer. Racemic doxapram was approximately 50% as potent as (+)-enantiomer, as would be predicted for a 50:50 mixture of active and inactive enantiomers.

TABLE 3

| Compound | doxapram | (−)-doxapram | (+)-doxapram |
|---|---|---|---|
| ED₅₀ (mg/kg IV) | 4.8 | >200 | 2.2 |

### Example 2

### Effect of (+)-Doxapram and (−)-Doxapram on Opioid-Induced Respiratory Depression in the Rat, as Determined by Plethysmography

All animal experiments were carried out according to the US law on animals care and use approved by Galleon Pharmaceuticals Institutional Animal Care and Use Committee (IACUC). Rats with pre-cannulated jugular vein (for administrating drugs) were acclimated to plethysmography chambers for a minimum of 60 minutes, or until animals were no longer restless. Each animal was dosed with morphine sulfate (10 mg/kg), dissolved in sterile water at a concentration of 10 mg/mL (supplied by Baxter Healthcare Corporation), via injection into the jugular vein catheter over a period of 5-10 seconds. After a period of 5 min, (−)-doxapram, (+)-doxapram, or racemic doxapram (1 mg/mL) was administered via infusion into the jugular vein at a rate of 0.020 mL/min for a 300 gram rat. Behavioral observations were made though the course of the experiment. After 20 min of infusion at 1 mg/kg/min, the infusion rates were tripled from 0.020 mL/min to 0.060 mL/min for all rats, based on body weight. After 20 minutes of infusion at this dose, the infusion pumps were turned off, and all animals were given a 20 minute recovery period, followed by a post-study analysis of rat health and behavior. The minute ventilation data indicate that (+)-doxapram significantly reverses opioid-induced respiratory depression in rat, whereas (−)-doxapram does not, as compared to vehicle. The small increase in minute ventilation seen towards the end of the experiment in the (−)-doxapram group was associated with behavioral toxicities and therefore cannot be distinguished from non-specific side effects. Results are illustrated in FIG. 2.

### Example 3

### Effect of (+)-Doxapram and (−)-Doxapram on Opioid-Induced Changes in Arterial Blood Gas Parameters in the Rat, as Determined by Arterial Blood Gas Analysis

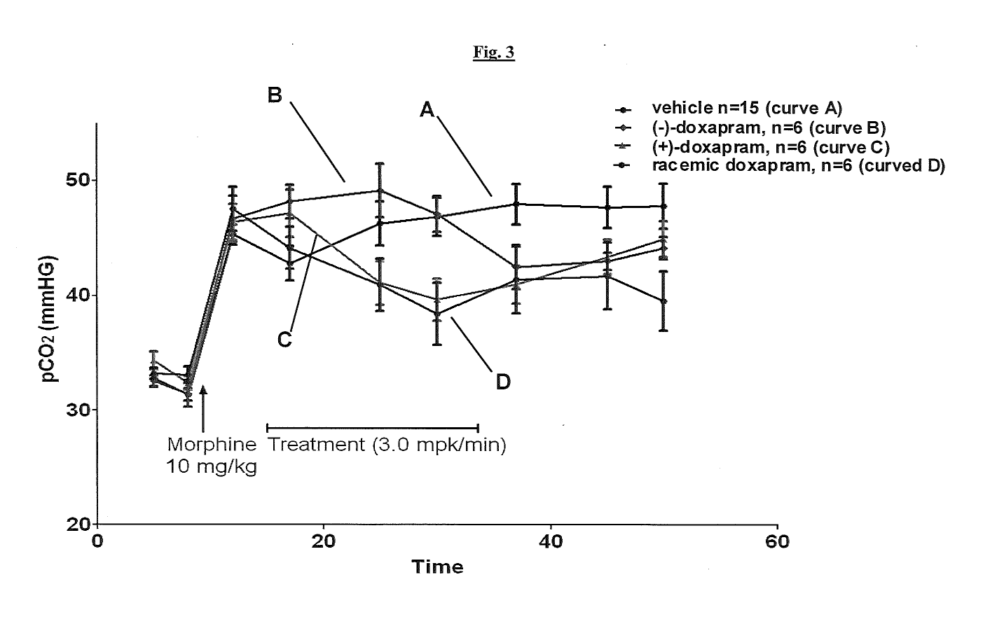

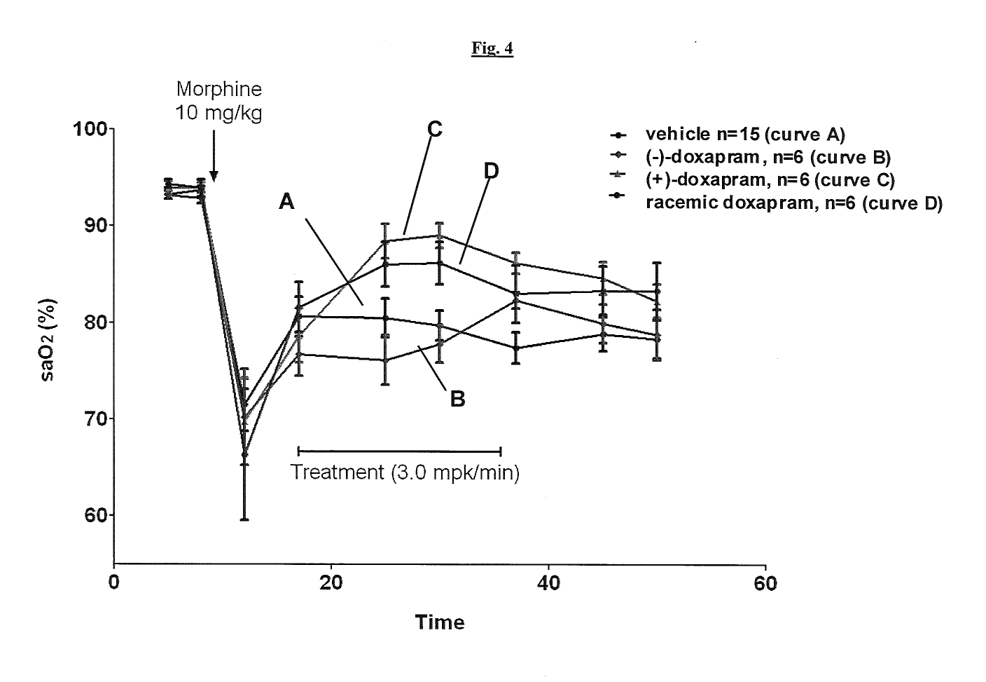

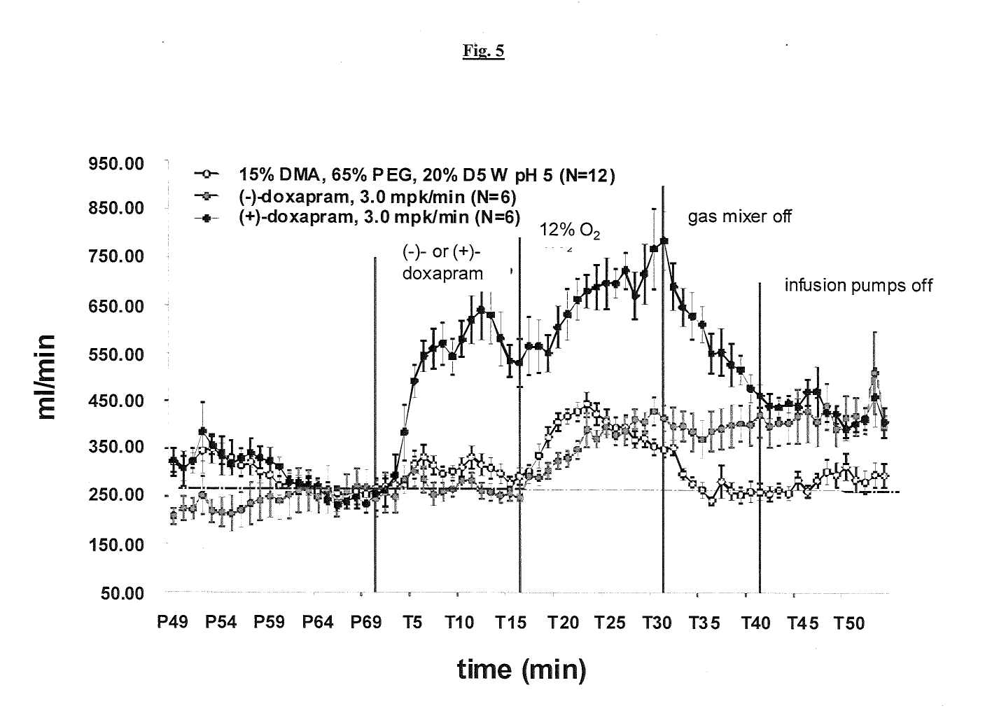

Rats with pre-cannulated jugular vein and femoral arterial catheters (for administrating drugs and obtaining blood samples respectively) were obtained from Harlan laboratories and kept at the animal facility at Galleon Pharmaceuticals until the experimental procedures. All animals experiments were carried out according to the US law on animals care and use approved by Galleon Pharmaceuticals IACUC. Each animal was dosed with morphine sulfate (10 mg/kg), dissolved in saline at a concentration of 10 mg/ml, via injection into the jugular vein over a period of 20 seconds with a 20 second flush of 0.9% NaCl saline. Prior to morphine administration, two 250 μL samples of arterial blood were aspirated from the femoral artery into a pre-heparinized syringe. The samples were analyzed on Radiometer's ABL Flex 800, where pO₂, pCO₂, pH, saO₂ and other parameters were recorded. Aspirated volumes of arterial blood were replaced by room temperature sterile saline (˜300 μL) slowly flushed back into the femoral arterial catheter of the rodent to prevent anemia and/or dehydration. Morphine was then administered and 2 minutes later another blood sample was taken. After a period of 5 min from the administration of morphine, (−)-doxapram, (+)-doxapram or racemic doxapram (15 mg/mL) was administered via infusion into the jugular vein at a rate of 60 μL/min/300 gram rat. The infusion started at t=15 minutes and ended at t=35 minutes. Arterial blood gas analysis occurred at time points t=17, 25, 30, 37, 45, and 50 minutes. The data show that (+)-doxapram and racemic doxapram significantly reverse opioid-induced respiratory depression in rat whereas (−)-doxapram does not, as compared to vehicle. The small improvements in blood gas parameters seen towards the end of the experiment in the (−)-doxapram group were associated with behavioral toxicities and therefore cannot be distinguished from non-specific side effects. Results are illustrated in Table 4 and FIGS. 3-4.

TABLE 4

| | | | PaCO₂: | PaO₂: | saO₂: |
|---|---|---|---|---|---|
| | Dose | pH | % | % | % |
| Compound | mpk/min | % reversal | reversal | reversal | reversal |
| Racemic doxapram | 3.0 | 58 | 90 | 22 | 33 |
| (−)-doxapram | 3.0 | −8 | 24 | −2 | −6 |
| (+)-doxapram | 3.0 | 49 | 79 | 41 | 41 |

* All parameters % reversal vs. composite vehicle group.
Effects measured 15 min into 20 min infusion of compound.
All studies: n = 6 rats.

### Example 4

### Effect of (+)-Doxapram, (−)-Doxapram and Racemic Doxapram on the Hypoxic Ventilatory Response in the Rat

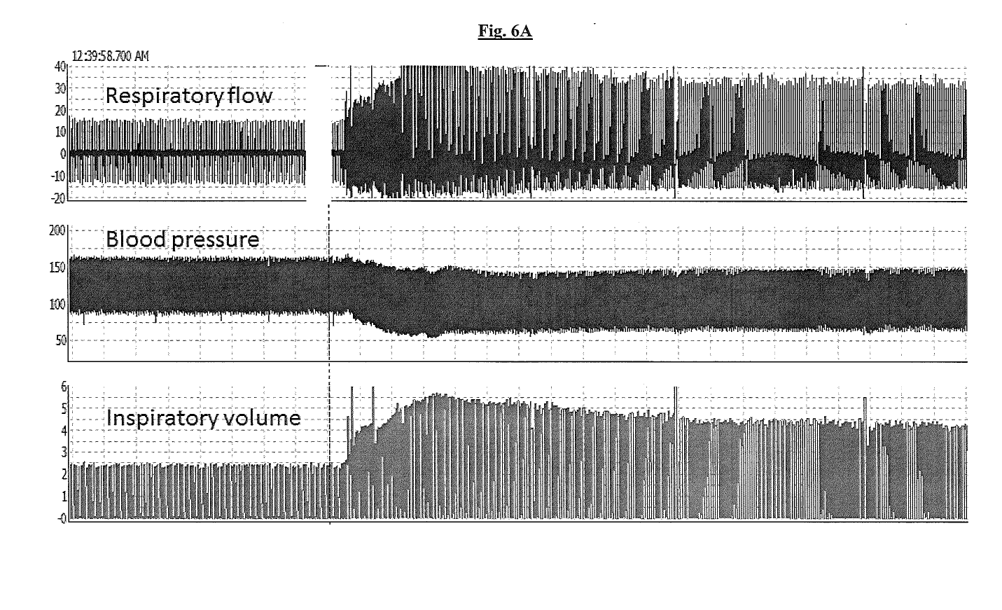

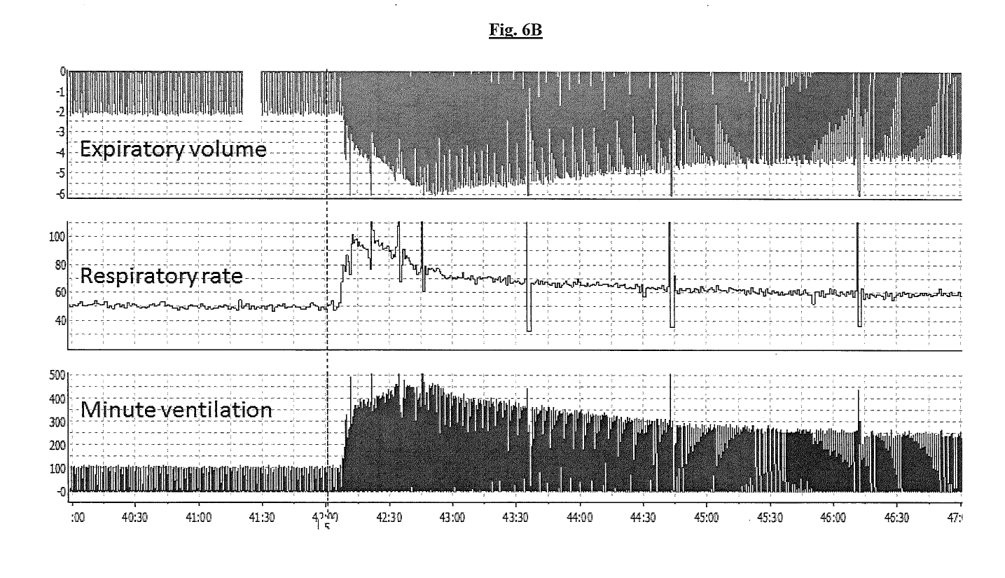

Rats with a pre-cannulated jugular vein (for administrating drugs) were acclimated to plethysmography chambers for a minimum of 60 minutes, or until animals were no longer restless. Each animal was dosed with (−)-doxapram, (+)-doxapram, or racemic doxapram via infusion into the jugular vein catheter over a period of 15 minutes, at 3 mg/kg/min at 0.020 ml/min based on a 300 gram rat. After a period of 15 minutes, an isocapnic, hypoxic mixture (12% O₂ balanced N₂) was administered into all chambers using a gas mixer (CWE inc. GSM-3 gas mixer) for 15 minutes. After 15 minutes, the gas mixer was turned off, resulting in normal room air pumped into the chambers. Ten minutes later, the infusion pumps were turned off, and all animals were given a 15 minute recovery period, followed by a post-study analysis of rat health and behavior. The data indicate that (+)-doxapram, and racemic doxapram significantly potentiated the hypoxic ventilatory response in the rat whereas (−)-doxapram did not, as compared to vehicle. The small increases in minute ventilation seen towards the end of the experiment in the (−)-doxapram group (i.e. T20-T55) were associated with behavioral toxicities and therefore cannot be distinguished from non-specific side effects. Results are illustrated in FIG. 5 and Table 5.

TABLE 5
Summary of results from Examples 1 through 5.

| | Assay/Rat Model | (−)- | (+)- |
|---|---|---|---|
| Assay | (IV dose) | doxapram | doxapram |
| Example 1 | In vivo cardio-respiratory study of | >200 | 2.1 |
| | dose-dependent increases in | | |
| | minute ventilation (MV) | | |
| | (ED₅₀ mg/kg IV bolus) | | |
| Example 2 | Plethysmography - | Not active | Active |
| | OIRD (MV) | | |
| | (1, 3 mg/kg/min) | | |
| Example 3 | Arterial Blood Gases - | Not active | Active |
| | OIRD (3 mg/kg/min) | | |
| | pCO₂ (mmHg) and sa O₂(%) | | |
| Example 4 | Plethysmography (MV) | Not active | Active |
| | Naïve and HVR | | |
| | (1, 3 mg/kg/min) | | |

Key:
OIRD: rat model of opioid-induced respiratory depression (10 mpk IV morphine)
Naïve: conscious rats without any other drug treatment or gas challenge
HVR: hypoxic ventilatory response elicited by 12% O₂

### Example 5

### Effect of (+)-Doxapram on Respiratory Flow, Respiratory Rate, Minute Volume and Blood Pressure in the Rat

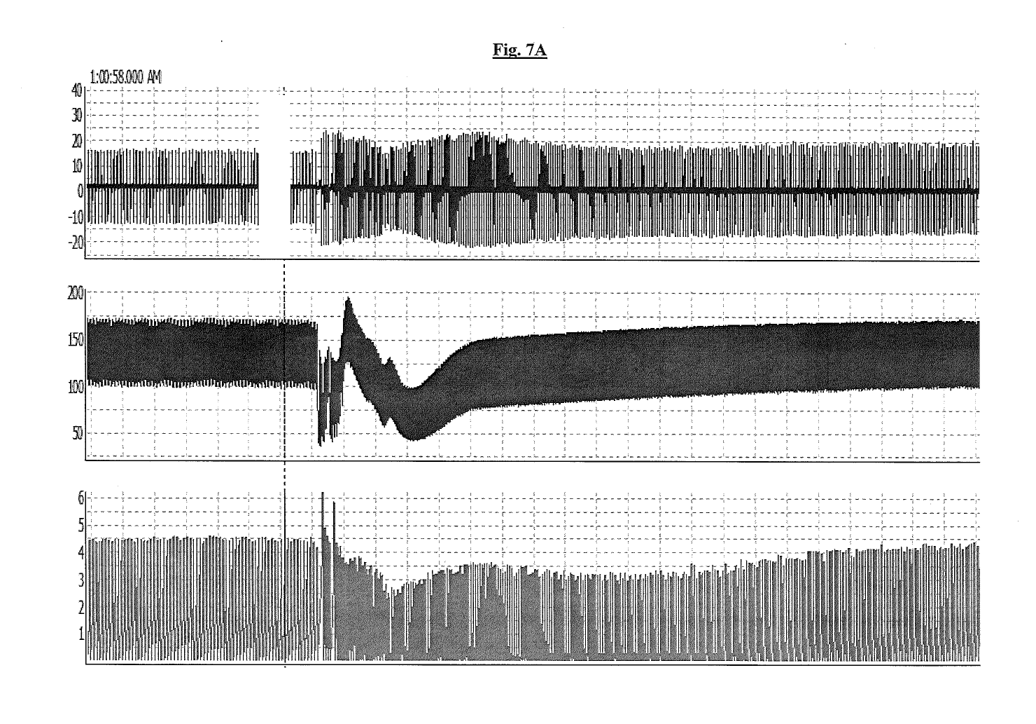

The procedure outlined in Example 1 was used herein. The data show that administration of 30 mg/kg IV (+)-doxapram increased respiratory flow, inspiratory and expiratory volume, as well as enhanced minute ventilation in rat. At this dose there was only a minimal reduction in blood pressure without associated arrhythmias. Results are illustrated in FIG. 6.

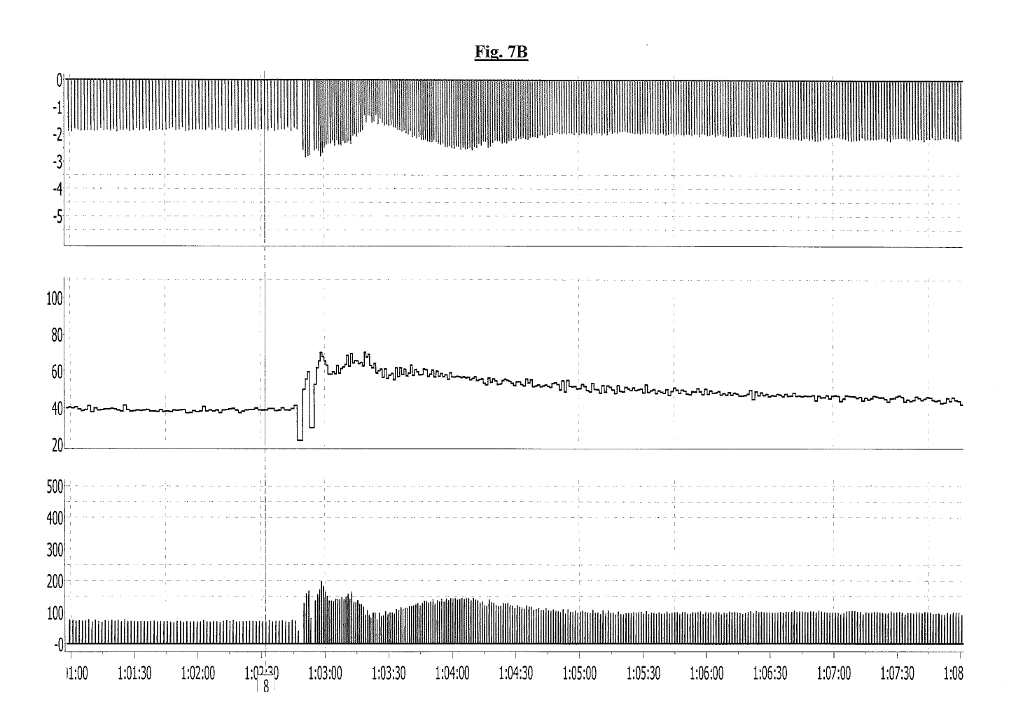

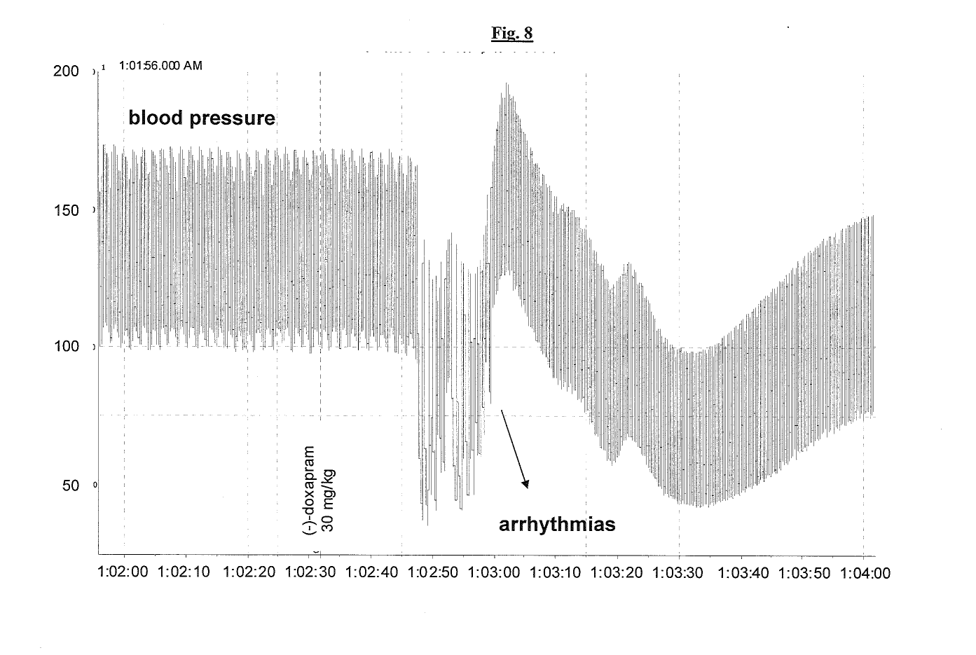

In contrast, administration of 30 mg/kg IV (−)-doxapram had only a minimal effect on respiratory flow, inspiratory and expiratory volume, and minute ventilation in rat. Results are illustrated in FIG. 7. Moreover, at this dose, (−)-doxapram caused a pronounced reduction in arterial blood pressure together with a period of associated arrhythmias (see Example 6 and FIG. 8).

### Example 6

### Effects of (−)-Doxapram in Blood Pressure in Rat

The procedure described in Example 1 was used herein to evaluate the effect of (−)-doxapram on mean arterial blood pressure (MAP) in the rat. As illustrated in FIG. 8, administration of 30 mg/kg IV (−)-doxapram, a dose that produced a minimal ventilatory response, caused a pronounced drop in blood pressure in rat, including a period of heart arrhythmias. Results are illustrated in FIG. 8.

### Example 7

### Observations of Animal Behavior and Adverse Effects

In Examples 1-7 described above, all rats are observed for behavioral changes and adverse effects, as recognized by those skilled in the art. Such effects include central nervous system and motor effects such as impairment, sedation, and convulsive potential, and mortality. Other effects related to bodily functions are also observable and may include changes in breathing, urination, defecation, posture, and normal cage activities (i.e., grooming, exploring, eating, etc.). Across the studies herein, it was observed that (−)-doxapram consistently produced a variety of adverse effects at the doses tested, whereas (+)-doxapram at the same doses did not. Table 6 illustrates these findings.

TABLE 6
Adverse Effects in Rats

| Assay/Model | Observed | (−)- | (+)- |
|---|---|---|---|
| (IV dose) | Adverse Event | doxapram | doxapram |
| Pleth. | Death | 5/6 | 0/6 |
| (30 mg/kg, IV bolus) | | | |
| Pleth. | Hunching, static, | 6/6 | 0/6 |
| (3 mg/kg/min) | increased urination & | | |
| | defecation | | |
| ABG post morphine | Clonic movements | 5/6 | 0/6 |
| (3 mg/kg/min) | | | |
| E-Phys | Cardiopulmonary | MAP ↓↓ | MAP ↓ |
| (≧10 mg/kg IV bolus) | response | Arrhythmias | |

Key:
E-Phys: in vivo electrophysiology;
ABG: arterial blood gases
Pleth.: whole body plethysmography;
MAP: mean arterial pressure
x/6: number of animals out of group of 6 that exhibited the adverse effect

### Example 8

### IV Pharmacokinetics

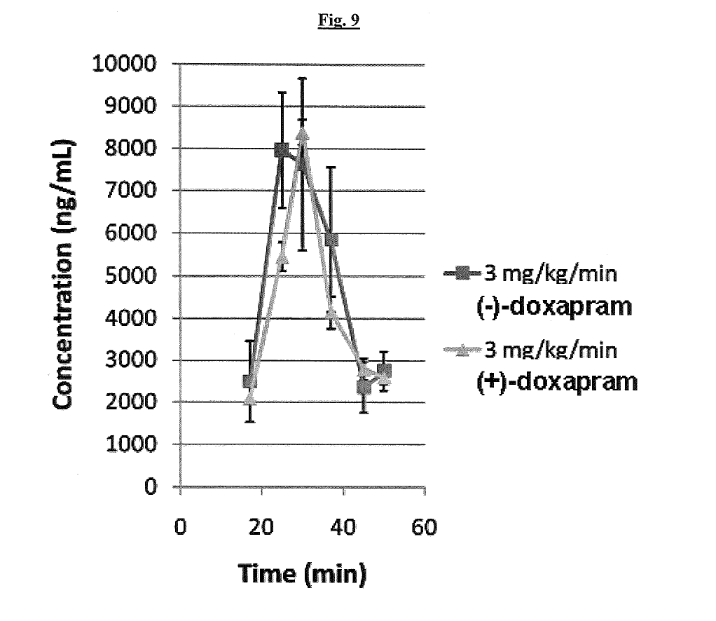

Comparative IV pharmacokinetics of a 20 min infusion of 3 mg/kg/min IV (+)-doxapram and (−)-doxapram showed that the plasma exposures of the two enantiomers are directly comparable in terms of time course, maximum concentration, exposure (AUC) and washout. The difference in efficacy and adverse events seen between the enantiomers is therefore due to genuine differences in the intrinsic pharmacodynamics (i.e. pharmacology and side effects profiles) of the enantiomers, as opposed to differential exposures/pharmacokinetics. Results are illustrated in FIG. 9.

The disclosures of each and every patent, patent application, and publication cited herein are hereby incorporated herein by reference in their entirety.

While the invention has been disclosed with reference to specific embodiments, it is apparent that other embodiments and variations of this invention may be devised by others skilled in the art without departing from the true spirit and scope of the invention. The appended claims are intended to be construed to include all such embodiments and equivalent variations.

What is claimed is:

1. A method of treating a subject in need of opioid therapy, wherein the opioid therapy produces or has the possibility of producing respiratory depression or a breathing control disorder in the subject, the method comprising administering to the subject an effective amount of a pharmaceutical composition comprising a compound selected from the group consisting of (+)-doxapram, a deuterated derivative of (+)-doxapram, any salt thereof and any combinations thereof, the method further comprising administering to the subject an effective amount of an opioid, wherein the composition is essentially free of (−)-doxapram, a deuterated derivative of (−)-doxapram, or any salt thereof.

2. The method of claim 1, wherein the compound is at least about 95% enantiomerically pure.

3. The method of claim 2, wherein the compound is at least about 97% enantiomerically pure.

4. The method of claim 3, wherein the compound is at least about 99% enantiomerically pure.

5. The method of claim 1, wherein the opioid comprises morphine, codeine, heroin, hydromorphone, hydrocodone, oxymorphone, oxycodone, meperidine, methadone, nalbuphine, butorphanol, buprenorphine, propoxyphene, pentazocine, dihydrocodeine, tapentadol, fentanyl, remifentanil, alfentanil, sufentanil, carfentanil, or any combinations thereof.

6. The method of claim 1, wherein the subject is further administered at least one additional compound selected from the group consisting of acetazolamide, almitrine, theophylline, caffeine, methyl progesterone, a serotinergic modulator, an ampakine, and any combinations thereof.

7. The method of claim 1, wherein the composition is administered in conjunction with the use of a mechanical ventilation device or positive airway pressure device on the subject.

8. The method of claim 1, wherein the composition is administered to the subject by an inhalational, topical, oral, buccal, rectal, vaginal, intramuscular, subcutaneous, transdermal, intrathecal or intravenous route.

9. The method of claim 1, wherein the administering of the compound takes place before or after the administering of the opioid to the subject.

10. The method of claim 9, wherein the administering of the compound takes place within 6 hours of the administering of the opioid to the subject.

11. The method of claim 1, wherein the compound is co-administered with the opioid to the subject.

12. The method of claim 11, wherein the compound is co-formulated with the opioid.

13. The method of claim 1, wherein the composition further comprises a pharmaceutically acceptable carrier.

14. The method of claim 1, wherein the subject is a mammal.

15. The method of claim 14, wherein the mammal is human.

16. A pharmaceutical composition comprising a pharmaceutically acceptable carrier, an opioid and a compound selected from the group consisting of (+)-doxapram, a deuterated derivative of (+)-doxapram, any salt thereof and any combinations thereof, wherein the composition is essentially free of (−)-doxapram, a deuterated derivative of (−)-doxapram or a salt thereof.

17. The composition of claim 16, wherein the compound is at least about 95% enantiomerically pure.

18. The composition of claim 17, wherein the compound is at least about 97% enantiomerically pure.

19. The composition of claim 18, wherein the compound is at least about 99% enantiomerically pure.

20. The composition of claim 16, wherein the opioid comprises morphine, codeine, heroin, hydromorphone, hydrocodone, oxymorphone, oxycodone, meperidine, methadone, nalbuphine, butorphanol, buprenorphine, propoxyphene, pentazocine, dihydrocodeine, tapentadol, fentanyl, remifentanil, alfentanil, sufentanil, carfentanil, or any combinations thereof.

21. A method of preventing or treating a breathing control disorder or disease in a subject in need thereof, the method comprising administering to the subject an effective amount of a pharmaceutical composition comprising a pharmaceutically acceptable carrier and a deuterated derivative of (+)-doxapram or a salt thereof, wherein the composition is essentially free of a deuterated derivative of (−)-doxapram or a salt thereof.

22. The method of claim 21, wherein the deuterated derivative of (+)-doxapram or a salt thereof is at least about 95% enantiomerically pure.

23. The method of claim 22, wherein the deuterated derivative of (+)-doxapram or a salt thereof is at least about 97% enantiomerically pure.

24. The method of claim 23, wherein the deuterated derivative of (+)-doxapram or a salt thereof is at least about 99% enantiomerically pure.

25. The method of claim 21, wherein the breathing control disorder or disease is selected from the group consisting of respiratory depression, sleep apnea, apnea of prematurity, obesity-hypoventilation syndrome, primary alveolar hypoventilation syndrome, dyspnea, hypoxia, and hypercapnia.

26. The method of claim 21, wherein the subject is further administered at least one additional compound useful for treating the breathing control disorder or disease.

27. The method of claim 26, wherein the at least one additional compound is selected from the group consisting of acetazolamide, almitrine, theophylline, caffeine, methyl progesterone and related compounds, a serotinergic modulator and an ampakine.

28. The method of claim 21, wherein the composition is administered in conjunction with the use of a mechanical ventilation device or positive airway pressure device on the subject.

29. The method of claim 21, wherein the subject is a human.

30. The method of claim 21, wherein the composition is administered to the subject by an inhalational, topical, oral, buccal, rectal, vaginal, intramuscular, subcutaneous, transdermal, intrathecal or intravenous route.

31. A method of preventing destabilization or stabilizing breathing rhythm in a subject in need thereof, the method comprising administering to the subject an effective amount of a pharmaceutical composition comprising a pharmaceutically acceptable carrier and a deuterated derivative of (+)-doxapram or a salt thereof, wherein the composition is essentially free of a deuterated derivative of (−)-doxapram or a salt thereof.

32. The method of claim 31, wherein the deuterated derivative of (+)-doxapram or a salt thereof is at least about 95% enantiomerically pure.

33. The method of claim 32, wherein the deuterated derivative of (+)-doxapram or a salt thereof is at least about 97% enantiomerically pure.

34. The method of claim 33, wherein the deuterated derivative of (+)-doxapram or a salt thereof is at least about 99% enantiomerically pure.

35. The method of claim 31, wherein the subject is further administered at least one additional compound useful for preventing destabilization of or stabilizing the breathing rhythm.

36. The method of claim 35, wherein the at least one additional compound is selected from the group consisting of acetazolamide, almitrine, theophylline, caffeine, a serotinergic modulator and an ampakine.

37. The method of claim 31, wherein the composition is administered in conjunction with the use of a mechanical ventilation device or positive airway pressure device.

38. The method of claim 31, wherein the subject is a mammal.

39. The method of claim 38, wherein the mammal is human.

40. The method of claim 31, wherein the composition is administered to the subject by an inhalational, topical, oral, buccal, rectal, vaginal, intramuscular, subcutaneous, transdermal, intrathecal or intravenous route.

41. A pharmaceutical composition comprising a pharmaceutically acceptable carrier and a deuterated derivative of (+)-doxapram or any salt thereof, wherein the composition is essentially free of a deuterated derivative of (−)-doxapram or a salt thereof.

42. The composition of claim 41, wherein the deuterated derivative of (+)-doxapram or any salt thereof is at least about 95% enantiomerically pure.

43. The composition of claim 42, wherein the deuterated derivative of (+)-doxapram or any salt thereof is at least about 97% enantiomerically pure.

44. The composition of claim 43, wherein the deuterated derivative of (+)-doxapram or any salt thereof is at least about 99% enantiomerically pure.
